(19) United States
(12) Patent Application Publication
(10) Pub. No.: US 2016/0121009 A1
(43) Pub. Date: May 5, 2016

(54) Optical Communication Systems and Methods

(71) Applicant: Woods Hole Oceanographic Institution, Woods Hole, MA (US)

(72) Inventors: Norman E. Farr, Woods Hole, MA (US); Clifford T. Pontbriand, North Falmouth, MA (US)

(21) Appl. No.: 14/947,859

(22) Filed: Nov. 20, 2015

Related U.S. Application Data
(63) Continuation-in-part of application No. 14/557,361, filed on Dec. 1, 2014, now Pat. No. 9,231,708.
Application No. 14/557,361 is a continuation-in-part of application No. 13/117,867, filed on May 27, 2011, now abandoned.
Application No. 13/117,867 is a continuation of application No. 11/348,726, filed on Feb. 6, 2006, now Pat. No. 7,953,326.
Application No. 14/557,361 is a continuation-in-part of application No. 13/344,430, filed on Jan. 5, 2012, now Pat. No. 8,953,944.
Application No. 13/344,430 is a continuation-in-part of application No. 13/940,814, filed on Jul. 12, 2013, now Pat. No. 9,235,048.
(60) Provisional application No. 61/430,081, filed on Jan. 5, 2011.
(60) Provisional application No. 61/671,426, filed on Jul. 13, 2012.

(51) Int. Cl. A61L 2/10; G02B 19/00

(52) U.S. Cl. CPC A61L 2/10; G02B 19/0019; G02B 19/0095

(57) ABSTRACT

A system and method to reduce fouling of a surface subjected to an aquatic environment with a light source. According to one aspect, an antifouling system including an LED for emitting UV radiation, one or more mounts for directing emitted UV radiation toward the surface, and control circuitry for driving the LED disposed in a watertight housing. According to another aspect, an antifouling system which employs a fluorescent lamp disposed within a pressure vessel including a UV-transmissive material to allow UV light to pass through the pressure vessel and reduce bio-fouling of any surface.

### CROSS-REFERENCE TO RELATED APPLICATIONS

This application is a continuation-in-part of (a) U.S. application Ser. No. 14/557,361 filed 1 Dec. 2014, which is a continuation-in-part of: (i) U.S. application Ser. No. 13/117,867 filed 27 May 2011, which is a continuation of U.S. application Ser. No. 11/348,726 filed 6 Feb. 2006, now U.S. Pat. No. 7,953,326; and (ii) U.S. application Ser. No. 13/344,430 filed 5 Jan. 2012, now U.S. Pat. No. 8,953,944; and is a continuation-in-part of (b) U.S. application Ser. No. 13/940,814 filed 12 Jul. 2013, and claims priority to U.S. Provisional Application Nos. 61/430,081 and 61/671,426 filed 5 Jan. 2011 and 13 Jul. 2012, respectively. The entire contents of each of the above-mentioned applications are incorporated herein by reference.

### STATEMENT REGARDING GOVERNMENT LICENSE RIGHTS

The invention described in U.S. application Ser. No. 13/940,814 filed 12 Jul. 2013 was made with U.S. government support under Grant Nos. OCE-0942835 and OCE-0737958 awarded by the U.S. National Science Foundation. The U.S. Government has certain rights in the invention.

### FIELD OF THE INVENTION

This invention relates to systems and methods to enhance optical signal transmission among a plurality of nodes within one or more amorphous broadcast media, including transmission that is subject to degradation by factors such as biological fouling of optical surfaces in at least one amorphous broadcast medium.

### BACKGROUND OF THE INVENTION

Sensor-bearing unmanned underwater vehicles (UUV), as well as cabled ocean observatories, have been deployed extensively to study both natural and man-made phenomena. Much of the wireless communication necessary for these activities is accomplished by acoustic communication systems. Such acoustic communication systems, however, are limited by low band-width and high latency, and do not permit video or other high-rate data transfers. Accordingly, improved underwater optical communication (opticom) systems have been developed such as those described by Fucile et al. in US Patent Publication No. 2005/0232638 and by Farr et al. in U.S. Pat. No. 7,953,326, the latter being incorporated herein by reference.

Opticom uses light instead of sound to carry information. An opticom system encodes a message into an optical signal, and then emits or transmits the optical signal from one communication node through a transmission medium to a receiver at another communication node, which reproduces the message from the received optical signal. The term “communication node” as used herein includes (i) movable opticom systems carried by non-stationary, mobile objects or entities such as a surface ship, a UUV, or a diver, and (ii) non-movable opticom systems at a stationary position such as within an underwater observatory. Advantages of opticom systems are identified for example in a News Release by Woods Hole Oceanographic Institution titled “Optical system promises to revolutionize undersea communications”, published Feb. 23, 2010.

While opticom systems provide high-band-width, bidirectional wireless underwater optical communications, their performance is subject to interference from light generated from secondary light-producing systems deployed within the nearby marine environment. Such interfering secondary lighting systems may include work site lights, photographic lighting, navigational lighting, directional lights, hand-held lights, beacons, and/or warning lights.

The growth and feeding of biofouling organisms, especially those which form a community on hard substrates, inhibits the operational characteristics of industrial objects such as lenses. Several approaches are used to address this problem, including applications of one or more anti-fouling coatings. However, in many circumstances a coating will not work. For example, windows of a submerged precision optical instrument cannot be coated due to concerns with obstructing the clarity of the windows, thereby affecting the instrument's measurements. Another approach is to remove the organisms manually, such as by scrubbing with wiping by a mechanism akin to a windshield wiper, but the use of mechanical components can increase the opportunities for failure and introduce additional complexity and cost into the system.

Maintaining an uncompromised visual connection through the window is particularly important in many communications systems. For example, scientists are deploying UUVs that, due to their mobility, can expand the reach of seafloor observatories. These UUVs typically carry sensors on-board and operate autonomously, carrying out pre-programmed missions. While certain types of UUVs are tethered by cable to the seafloor observatories, the tethered UUVs have a short range of motion and are limited by the length of the tether. Scientists are also deploying un-tethered UUVs which may be controlled wirelessly by an acoustic communication system or an optical communication system. Acoustic communication systems, however, tend to be limited by low bandwidth and high latency, and do not permit video or other high-rate data transfers.

Accordingly, there is a need to provide an antifouling device that prevents and/or removes organisms from an optical surface in a marine environment or other amorphous broadcast medium. There is also a need for such a device to remove the organisms from a window while maintaining the integrity of the window for accurate sensor readings and communications. It is also desirable to mitigate many typical light interference issues that would otherwise degrade optical communication signals in at least one amorphous medium.

### SUMMARY OF THE INVENTION

An object of the present invention is to improve optical communication among a plurality of communication nodes in at least one amorphous medium of a gas such as air, of a liquid such as water, and/or a vacuum.

Another object of certain aspects of the present invention is to reduce fouling of a surface of an optically transparent element utilizing a light source. By using LEDs in certain embodiments, such a system may be more efficient, have a longer lifetime, and be more compact than traditional systems. The systems and methods may be further augmented by varying wavelengths and duty cycle.

Yet another object of the present invention is to combine optical antifouling with optical communication among a plurality of communication nodes.

This invention features a system and method to reduce fouling of a surface subjected to an aquatic environment with a light source. According to one aspect, an antifouling system including an LED for emitting UV radiation, one or more mounts for directing emitted UV radiation toward the surface, and control circuitry for driving the LED disposed in a watertight housing. According to another aspect, an antifouling system which employs a fluorescent lamp as the source of antifouling radiation which is disposed within a pressure vessel including a UV-transmissive material to allow UV light to pass through the pressure vessel and reduce bio-fouling of any surface.

In accordance with one embodiment, the optically transparent element is a window or a lens. The emitted UV radiation may have a wavelength between about 240 nm and about 295 nm, in one embodiment between about 250 nm to 260 nm. The antifouling light source may be disposed in a watertight enclosure, which may have a UV transparent port and retains a gas selected from atmospheric air, an inert gas, nitrogen gas, and combinations thereof. In other embodiments, the mount may be disposed on a side of the optically transparent element remote from the surface, and the optically transparent element may be made of a UV transparent material. In additional embodiments, the control circuitry is adapted to maintain a constant duty cycle of the LED, which may be at least about 10%. An attenuated dosage reaching the surface may be at least about 0.5 kJ/m². A kill efficiency at the surface may be at least about 95%.

In some embodiments, the system includes an end cap adapted to provide mechanical and electrical connections to an object such as a node, an observatory, a transducer, an optical modem, a vehicle, an AUV, and ROV, an UUV, a winch, a dock and a profiler. In one embodiment, the system further includes a configurable optical reflector capable of tailoring the UV emission to at least one of a wider angle pattern and a narrower angle pattern relative to the first pattern of the emission. In certain embodiments, the surface is a cable, a winch, a spool, a fin, a propeller, a light, a sensor, a transducer, an optically transparent surface, a window, a camera window, a lens, or a surface unsuitable for an antifouling coating. In one embodiment, the system is capable of reducing biofouling of the surface when disposed a distance from the surface of at least 30 cm.

One or more antifouling features are combined in some embodiments with a system that broadcasts an optical signal through an amorphous medium to a detector, also referred to as a receiver. The system includes a primary emitter capable of producing a primary optical signal having a first intensity during at least one broadcast period and capable of transmitting the primary optical signal through the amorphous medium.

In some embodiments, the antifouling light source emits radiation at a second intensity and is controlled by a controller as a secondary emitter, and the controller modulates the second intensity of the secondary emitter in synchrony with the primary optical signal. In certain embodiments, the resultant signal is of higher intensity than either the intensities of the primary signal or the secondary signal alone. In one embodiment, the controller changes the timing of the secondary emission relative to primary emission, such as by delaying the emitter that is closer to the detector to achieve substantially simultaneous reception by the detector of both signals. In a number of embodiments, the secondary emitter is normally operated at a duty cycle of at least 50 percent, typically at least 75 to 100 percent, when activated. In one embodiment, the secondary emitter is at least one of a work site light, a light to illuminate a photographic subject, a light on a submersible vehicle, a hand-held light, and a beacon.

In some embodiments, the controller includes at least one of a multiplexer and a signal splitter. In certain embodiments, the system further includes at least one receiver such that the system is capable of bidirectional communication with a third, remote emitter. In one embodiment, the second emitter is suppressed when the receiver senses that the third emitter is transmitting.

### BRIEF DESCRIPTION OF THE DRAWINGS

In what follows, preferred embodiments of the invention are explained in more detail with reference to the drawings, in which:

FIG. 1 is a schematic diagram of a system according to the invention immersed in an amorphous medium M and broadcasting to a receiver R;

FIG. 2 is a schematic diagram of a system according to the invention carried by an underwater vehicle and in communication with a seafloor observatory;

FIG. 3 is a more detailed block diagram of a primary emitter according to one embodiment of the invention; and

FIG. 4 is a more detailed block diagram of a receiver utilized according to one embodiment of the invention;

FIG. 5 depicts a network architecture for an underwater communication system according to one embodiment of the invention;

FIG. 6 illustrates an underwater optical communication network including a plurality of underwater optical modems and underwater vehicles according to an embodiment of the invention;

FIG. 7A is a schematic, perspective, semi-transparent view of an optical modem, in accordance with one embodiment of the invention;

FIG. 7B is a schematic plan view of the optical modem of FIG. 7A;

FIG. 7C is a schematic cross-sectional view of the optical modem of FIG. 7A taken along line C-C in FIG. 7B;

FIG. 8 is a schematic diagram of a timer circuit for use with the optical modem of FIG. 7A, in accordance with one embodiment of the invention;

FIG. 9 is a schematic side view of an experimental setup for testing the effectiveness of UV LEDs, in accordance with one embodiment of the invention;

FIG. 10 is a schematic perspective view of a UV lamp device according to one embodiment of the present invention;

FIG. 10A is a schematic end view of the device illustrated in FIG. 10 and FIG. 10B is a cross-sectional side view along lines B-B of FIG. 10A;

FIG. 10C is a schematic cross-sectional side view similar to FIG. 10B showing an optical reflector;

FIG. 10D is a schematic end view of the device illustrated in FIG. 10C;

FIG. 10E is a schematic end view of an optical reflector showing the angle between opposing walls of the reflector;

FIG. 11 is a schematic diagram of divers communicating underwater with each other, with an UUV and a buoy, with one or more links to an airplane;

FIG. 12 is a view similar to FIG. 11 with some of the divers also communicating above the surface of the water;

FIG. 13 is a view similar to FIG. 11 showing communication with a shore-based node such as a human interacting with one or more of the UUV, the divers and the airplane;

FIG. 14 is a schematic diagram of a diver node having both an optical modem and an acoustic modem;

FIG. 15 is a schematic diagram of a buoy node including multiple optical transceivers;

FIG. 16A is a schematic diagram depicting one construction of the data pathways for optical-only link and FIG. 16B depicts an acoustic-only link, with video traffic automatically halted in acoustic-only mode;

FIG. 17 illustrates sunlight measurement with unfiltered receivers at upward and downward orientations with optical power by depth, according to one construction;

FIG. 18 depicts three absorptive glass filter spectral transmissions overlaid with three LED emitters showing percent transmission by wavelength, according to one construction;

FIG. 19 depicts the measured optical power at upward-facing and downward-facing receivers (signal and solar background) versus depth in a 15-m separation test, according to one construction; and

FIG. 20 shows the measured optical power at upward-facing and downward-facing receivers (signal and solar background) versus depth in a 25-m separation test, according to one construction.

### DETAILED DESCRIPTION OF THE PRESENTLY PREFERRED EMBODIMENTS

This invention may be accomplished by a system and method to reduce fouling of a surface subjected to an aquatic environment with a light source. According to one aspect, an antifouling system including an LED for emitting UV radiation, one or more mounts for directing emitted UV radiation toward the surface, and control circuitry for driving the LED disposed in a watertight housing. According to another aspect, an antifouling system which employs a fluorescent lamp as the source of antifouling radiation which is disposed within a pressure vessel including a UV-transmissive material to allow UV light to pass through the pressure vessel and reduce bio-fouling of any surface.

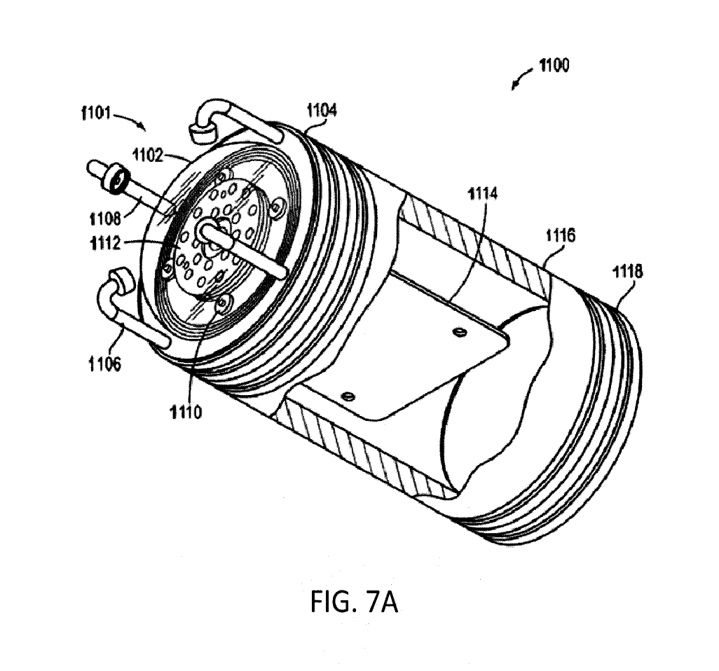

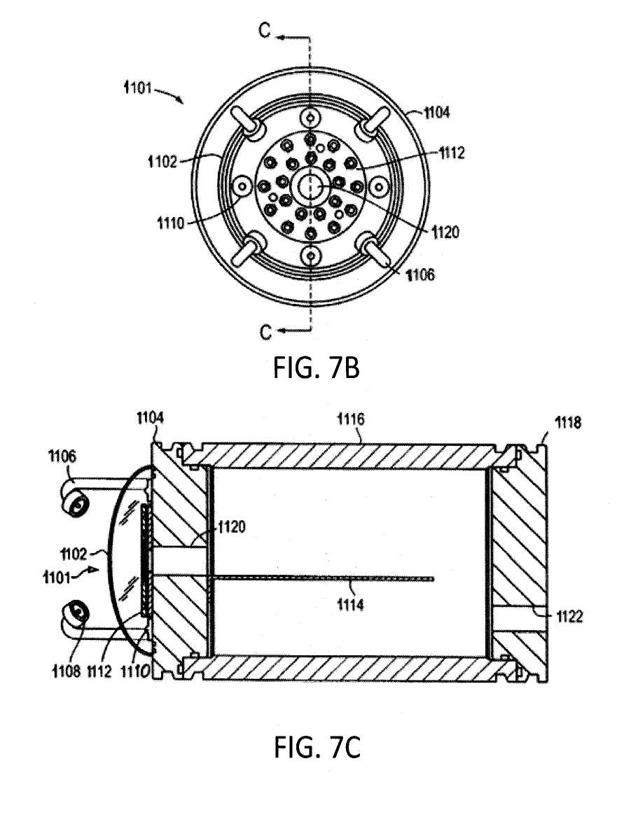

Examples of systems and methods according to the present invention for treating surfaces such as optical elements are described below in particular regarding FIGS. 7A-10B. Various novel optical communication systems that may include antifouling capabilities are described below in relation to the other Figures.

As described in U.S. application Ser. No. 14/557,361 filed 1 Dec. 2014, which is incorporated herein by reference, submerged optical communication (“opticom”) systems must often operate in the presence of secondary lighting (e.g. from the illumination of a work site). In order to minimize the impact of the secondary lighting on detector performance (e.g. output deterioration from the detector), and increase the effective signal intensity reaching the detector, some constructions of the current invention provide for entrainment of the light intensity from secondary light sources to the pattern of signals emanating from the opticom emitter. Entrainment causes the background signal produced from the secondary lighting to: (i) no longer be constant and (ii) become in effect, a secondary emitter, transmitting and reinforcing the same signal pattern as the primary emitter. Particularly benefited are those submergible opticom systems meant for operation in dark water which employ a detector that is negatively impacted (e.g. reduced signal to noise level) by the presence of a sustained background light.

The invention improves opticom transmission systems comprising signal detectors, also referred to as receivers, which are subject to output degradation from background signals within the amorphous broadcast medium. More specifically, some constructions of the invention entrain specific sources of optical background signal to the output pattern of the primary signal emitter, thereby enhancing the signal reaching the detector positioned within the amorphous medium.

Suitable primary emitters can be any device capable of producing a signal to be transmitted through the broadcast medium to a detector, wherein the transmitted signal or the act of transmitting the signal can be used by a signal processor to entrain the output of a source of a background signal of the same modality. In preferred embodiments, the primary emitter is an LED or array of LEDs. In the most preferred embodiments the primary emitter emits light in the visible range, preferably encompassing wavelengths within the blue color range. The light may be a mixture of wavelengths such as white light or it may be monochromatic. The characteristics of the optical signal to be transmitted through the broadcast medium to the detector are those known to practitioners of ordinary skill and are exemplified by Farr et al. in U.S. Pat. No. 7,953,326, incorporated herein by reference.

The detector is selected for its compatibility with the emitter, and its ability to detect the signal emitted therefrom. In general, the detector will have the capability of converting received light originating from the emitter to an electrical output. In some cases the detector is a photomultiplier tube (“PMT”), or the like. PMTs are capable of sensing single photon events and their sensitivity can be controlled by changing the voltage used to power the tube. In the most preferred embodiments, the detector is a PMT designed with the largest angular reception possible so that it most preferably is capable of detecting emitted light arriving from at least a hemispherical area.

Detectors comprising PMT's may benefit most from the invention, since substitution of a steady state light beam from a secondary emitter with the inventive beam of fluctuating intensity will minimize corresponding gain reduction in the PMT, while at the same time enhancing the overall signal received due to signal reinforcement by the entrained secondary emitter signal.

Without entrainment, a non-modulated secondary light source behaves as a noise source to the detector leading to a reduction in the maximum operating range of the emitter-detector system. The degree to which range reduction actually occurs is dependent on the ratio of non-modulated to modulated light received. In a configuration where a receiver collects light from two optical sources of equivalent power where one is modulated and other non-modulated, there is the equivalent of an 8 dB decrease in power compared to the primary modulated source alone. Submerged opticom systems are most susceptible to deteriorated response when non-modulated sources are located near the receiver because the amount of received light can be many times greater than the power received from a remote transmitter. A typical illumination source located near the receiver can reduce overall link range by more than 97%. Use of the inventive entrained secondary emitters as compared to non-modulated secondary emitters are expected to improve the signal to noise level of the detector by at least 1% to 2%, preferably 1% to 5%, and in most embodiments 1% to 10%.

The secondary emitter produces a signal of the same modality (e.g. light) as the primary emitter. When in operation, the output of the secondary emitter is of a wavelength composition, and format that it can be detected by the detector and therefore is a potential source of background signal, noise or interference. Furthermore, if operated as a steady output (i.e., as a non-entrained signal), the light from the secondary emitter, might lead to degradation of the performance/sensitivity of the detector. The inventive approach, however, reduces many or all of these negative effects of the secondary emitter on system performance.

In most instances the purpose of the secondary emitter is independent from that of the primary emitter. That is, while the primary emitter is intended to relay a signal to the detector for communication purposes, the secondary emitter is generally used to provide a lighting function. Typical lighting functions for a secondary emitter include: lighting a work site, illuminating a photographic target or subject, serving navigational purposes on a submersible vehicle, directional lighting, operating lights, a hand held light, a beacon, a work light, and a warning light.

In general, the emitted light from the secondary emitter will contain at least one wavelength capable of passing through the broadcast medium with an acceptable level of attenuation, such that it will travel the desired distance and arrive at the detector with an intensity that is detectable by the detector.

Light wave lengths between 400 nm and 500 nm pass through water with less attenuation than most other wavelengths and will generally be present in the emitted light. Most of the constituent wavelengths, when white light is passed through a long water path length, are more rapidly attenuated by the water than wavelengths in the 400 nm to 500 nm range. Therefore for the greatest optical telemetry range (e.g. 100 m to 200 m), it is most efficient to use light comprising wavelengths in the 400 nm to 500 nm “window”. For color imaging, which takes place at much shorter ranges (e.g. 10 m), white light is required.

An optical communications system when exposed to a non-modulated secondary light source undergoes a reduction in the maximum operating range due to a reduction in sensitivity of the detector. The range reduction is dependent on the ratio of non-modulated to modulated light received. Synchronizing or entraining a secondary light source to the primary emitter, has the opposite effect and increases operational range of the overall system.

To achieve effective entrainment, the secondary illumination source(s) preferably are synchronized to within 95% of the primary communications source. In a configuration where a receiver collects power from two optical sources of equivalent power synchronizing the second source adds approximately 4 dB to the primary signal for a total improvement of 6 dB. In two-emitter systems operating under optical conditions that would support a maximum range of 50 meters, entrainment will result in a range enhancement of at least 2, preferably 5, 10, 25, or up to 30 or 50 meters.

Suitable emitters (both primary and secondary) should be capable of rise and fall times of less than 1 microsecond, preferably less than 50 nanoseconds, more preferably less than 1 nanosecond, and ideally less than 10-100 picoseconds. Current LEDs operate in the greater than 100 picosecond range; to achieve rates of less than 100 picoseconds, laser-based emitters will generally be employed.

The combined circuitry elements of the secondary emitter must be capable of producing a modulating light beam synchronized to within 10 nanoseconds of the modulated beam of the primary emitters. Specifically, the circuitry will be assembled such that the entrained signal of the secondary emitter is substantially identical to that of the primary emitter with a following delay of no greater than 10-50 nanoseconds, preferably 1 nanosecond, and most preferably less than or equal to 100 picoseconds. The circuitry must also operate with less than 1 nanosecond of jitter.

Generally, information processing for the emitter and detector is accomplished through half-duplex multiplexing. The multiplexing frame rate is generally from 1 HZ to 5 Hz often 100-200 Hz, and in some embodiments up to 1000 Hz. In one embodiment, optimal optical performance of the detector is achieved by using light and secondary emitters that are synchronized to the primary emitter both in modulation rate and time division multiplexing.

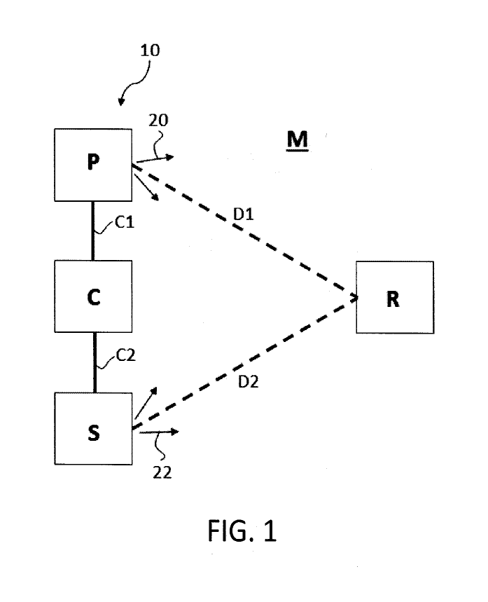

In most embodiments, the primary and secondary emitters are approximately the same distance from the receiver or modem. System 10, FIG. 1, illustrates one construction of a system according to the present invention having a primary emitter P, a controller C, and a secondary emitter S that are immersed in an amorphous medium M and spaced from a receiver R, also referred to as detector R. Controller C has a first communication link C₁ with the primary emitter P and a second communication link C₂ with the secondary emitter S. In some constructions, at least one of links C₁ and C₂ is a wire that carries electrical signals; in other constructions, at least one of links C₁ and C₂ is a wireless communications link as is known by those of ordinary skill in the field. One known advantage of wireless communications is that it is generally easier to reconfigure wireless components of a system. Primary emitter P, which generates primary optical signals as indicated by arrows 20, is at a distance D₁ from receiver R and secondary emitter S, which generates secondary optical emissions as indicated by arrows 22, is at a distance D₂. As described in more detail below, in some constructions the primary emitter P and/or the receiver R are optical modems that are capable of both transmitting and receiving optical communication signals.

In certain preferred configurations, the distance D₁ of the primary emitter P to the receiver R is within 25 ft of the distance D₂ of the secondary emitter S to the receiver R; in other embodiments, the absolute value of D₁−D₂ is less than 20, 15, 10, or 5 feet. When observing such distance differentials is not possible, then in certain constructions a delay function is included in the circuitry of the controller or one or both emitters, in order to more effectively synchronize the signals reaching the detector (receiver R) by adjusting the actual emission timing of one or more emitters.

Effective transmission of optical data between the inventive emitters and detectors will vary in distance and rate depending on water clarity. In substantially clean water, the inventive emitter/detector systems will transmit up to 110 meters at data rates of 2, 5, 8, 10, or 12 megabits/second (Mbps). To achieve transmission distances of 200 meters in clean water transmission rates of less than 2, 1.5, 1.0, 0.75, or 0.5 Mbps will be needed.

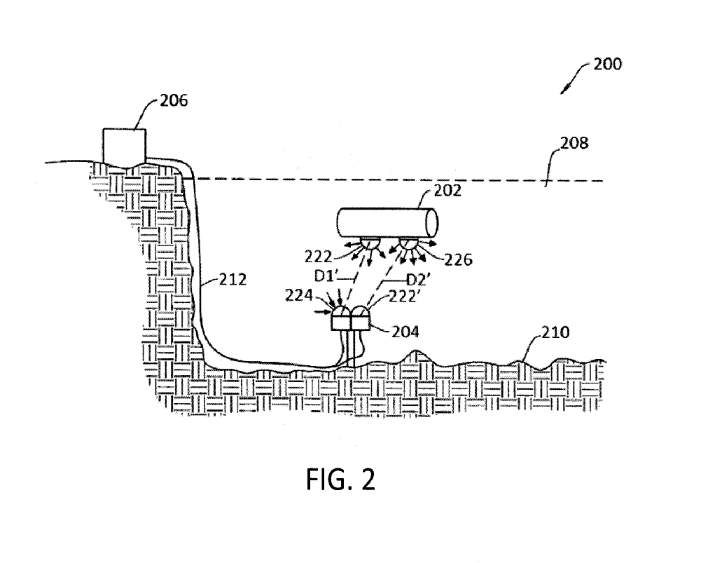

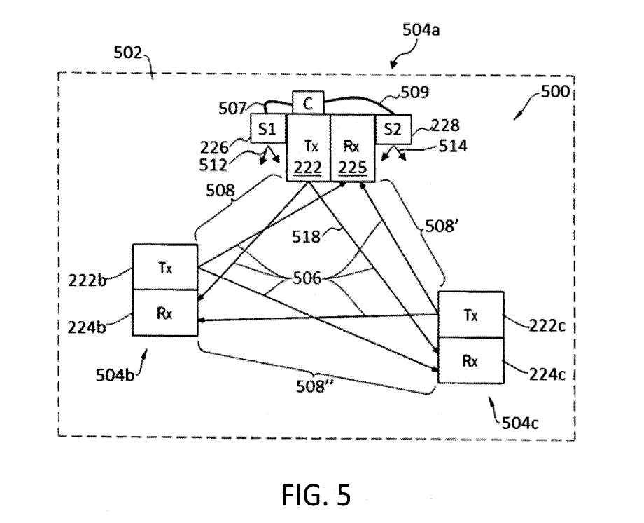

System 200 according to the invention, FIG. 2, is carried by an underwater vehicle 202 that is in communication with a seafloor observatory 204 immersed in ocean 208 and positioned on seafloor 210. Vehicle 202 includes a primary transmitter 222 and a secondary emitter 226 such as an illumination light. In this construction, observatory 204 includes a receiver 224 and is connected by cable 212 with a land unit or station 206. As illustrated in FIG. 2, primary transmitter 222 is at distance D₁′ from receiver 224 and secondary emitter 226 is at distance D₂′. In some constructions, observatory 204 also includes a transmitter 222′ and underwater vehicle 202 includes a receiver 225 as described in more detail below in relation to FIG. 5. Vehicle 202 communicates with cabled observatory 804 using a communication protocol, e.g., time division multiple access (TDMA), code division multiple access (CDMA), space division multiple access (SDMA), frequency division multiple access (FDMA) or any other suitable communication protocol. The description of underwater unmanned vehicle 802 and seafloor observatory 804 by Farr et al. in U.S. Pat. No. 7,953,326 is expressly incorporated herein by reference.

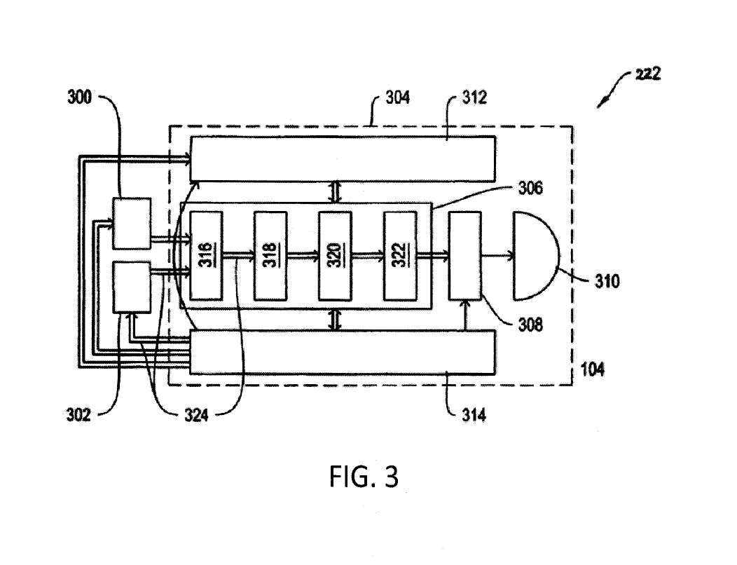

FIG. 3 is a more detailed block diagram of a primary emitter 222 according to one embodiment of the invention connected to input devices including a data element 300 and a control element 302, which provide an input signal containing information to be transmitted. The primary emitter 222, also referred to as transmitter 222, receives input signals from an input device and then converts the format of the input signal to a format that can be used to transmit the information contained in the input signal through seawater or other communication medium. In one embodiment, the primary emitter 222 is configured to receive input signals from different types of input devices. In such an embodiment, the input devices may include data elements such as sensors including a temperature sensor, a pressure sensor, a motion sensor such as an acoustic sensor and/or a seismic motion sensor, a light sensor, and/or a video camera. The primary emitter 222 includes a water-proof enclosure 304 that houses a microprocessor 306, an oscillator 308, a directional element 310, a memory 312 and a power supply 314. The microprocessor 306 includes a data interface module 316, a protocol/buffer module 318, a coding module 320 and a modulating module 322. Elements are electrically connected to each other by interconnect bus 324.

Data element 300 includes sensors that typically acquire information from the surrounding environment such as temperature, pressure, gaseous composition, vibrations or other motion, and/or visual appearance. In one embodiment, a data element 300 includes at least one of a temperature sensor, a moisture sensor, a pressure sensor, a gas sensor, a light sensor, a motion sensor, and a video camera. In another embodiment, the data element 300 may include a laser induced breakdown spectrometer, Raman spectrometer or mass spectrometer. The data element 300 may include other devices that collect information from the surrounding environment, for example at least one type of electromagnetic emission, such as optical radiation or narrow-band EM field, and/or at least one type of mechanical wave emission, such as ground-coupled vibration, sonic, ultrasonic, or low-frequency (infrasonic) acoustic emissions, for marine-based and/or terrestrial alternate-energy sources or other installations or human activity. The data element 300 typically generates a data signal that contains information sensed from the surrounding environment. The data signal generated by the data element may include electrical DC or AC signals having characteristics representative of the information collected. For example, the amplitude of a DC electrical signal may be representative of the temperature of the surrounding environment. In one construction, input signals are obtained from MEMS (Micro-Electro-Mechanical Systems) accelerometers to sense ground motions or other vibrations such as described by Cochran et al. In “A Novel Strong-Motion Seismic Network for Community Participation in Earthquake Monitoring, IEEE Instrumentation & Measurement Magazine, December 2009, pages 8-15. Other suitable input devices for sensing at least one ocean parameter are disclosed in U.S. Pat. No. 5,894,450 by Schmidt et al., U.S. Pat. No. 7,016,260 by Bary, and U.S. Pat. No. 7,711,322 by Rhodes et al., for example.

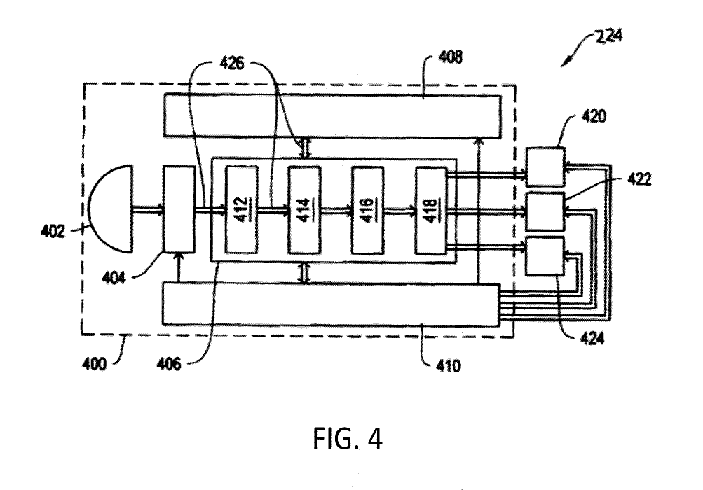

FIG. 4 is a more detailed block diagram of a receiver 224 utilized according to one embodiment of the invention having a waterproof enclosure 400 that houses a directional element 402, a detector 404, a microprocessor 406, a memory 408 and a power supply 410. The microprocessor 406 includes a demodulating module 412, a decoding module 414, a protocol/buffer module 416 and a device interface module 418. The receiver 224 is connected to output devices such as a computer 420, a data element 422, or an analog element 424. Components are electrically connected to each other by interconnect buses 426. The description of transmitter 102 and receiver 104 by Farr et al. in U.S. Pat. No. 7,953,326 is expressly incorporated herein by reference for transmitter 222 and receiver 224 for the present invention.

In a number of constructions, the detector 404 receives the transmitted signal from the directional element 402 such that the information in the transmitted signal is processed by electronics in the receiver 224 as well as outside of the receiver 224. As an example, in optical communication where the transmitted signal is the optical wavelength range of the electromagnetic spectrum, the detector 404 is configured to detect the optical transmitted signal and convert the signal to an electrical signal so that the electronics in the microprocessor 406 may process the information in the transmitted signal. In one embodiment, the detector 404 is configured to detect electromagnetic waves in the optical spectrum. In one such embodiment, the detector 404 includes a photomultiplier tube (PMT). In other embodiments the detector 404 may include at least one of a charge coupled device (CCD), a CMOS detector and a photodiode. PMTs typically provide higher sensitivity and lower noise than photodiodes. The spectral response of the bialkali PMTs typically peak in the blue wavelength range with a quantum efficiency of about 20%. Their gain is typically on the order of 10⁷. In certain embodiments, the detector 404 is formed together with the directional element 402. As an example, hemispherical PMTs such as the HAMAMATSU® R5912, as available by February 2006, combine hemispherical directional element 402 with a detector 404. The detector 404 sends the detected signal (typically a value of electrical current corresponding to the intensity of the received electromagnetic radiation) to a demodulating module 416.

In some constructions, in addition to buffering and protocol adjustment capabilities, the protocol/buffer module 416 also includes buffer circuits that are configured to amplify the decoded signal from the decoding module 414. Further, in certain constructions the receiver 224 also includes an Automatic Gain Control (AGC) module that controls the received power of the signal so that the received power is maintained fairly constant for different ranges. In particular, the AGC limits the power of the received signal transmitted over a short distance.

FIG. 5 depicts a network architecture of a network 500 according to the present invention within an underwater medium 502 including an underwater communication system 504a according to one embodiment of the invention communicating with optical modems 504b and 504c having transmitters Tx, components 222b and 222c, and receivers Rx, components 224b and 224c, respectively. In this construction, system 504a includes a transmitter Tx 222, a receiver Rx 225, secondary optical emitters S1 and S2, illustrated as light sources 226 and 228, respectively. A controller C synchronizes emissions 512 and 514 from emitters S1 and S2 with transmitter 222 via connections 507 and 509 so that enhanced optical signals 516 and 518 reach receivers 224b and 224c, respectively. The transmitters Tx and the receivers Rx send and receive information from each other along the direction of arrows 506. System 504a is separated by optical modems 504b and 504c by distances 508 and 508′, respectively, while modems 504b and 504c are separated by distance 508”.

In some constructions, at least one of system 504a, optical modem 504b, and optical modem 504c are mobile, and distances 508, 508′ and/or 508″ vary according to positioning of those units by one or more users, by currents within medium 502, or by other factors which alter their spatial relationships. In some embodiments, establishing the optical data connection between system 504a and units 504b and 504c includes determining acceptable optical ranges for distances 508, 508′ and 508″, respectively. In some embodiments, an optical communication network 500 is extended by disposing a third optical modem within an optical range of modem 504b, and disposing a fourth optical modem within an optical range of modem 504c.

The systems and methods described herein can be utilized to provide a reconfigurable, long-range, optical modem-based underwater communication network. In particular, the network provides a low power, low cost, and easy to deploy underwater optical communication system capable of being operated at long distances. Optical modem-based communication offers high data rate, and can be configured to generate omni-directional spatial communication in the visual spectrum. The omni-directional aspect of communication is advantageous because precise alignment of communication units may not be required. The optical modems may be deployed by unmanned underwater vehicles (UUVs) and physically connected by a tether (e.g., a light-weight fiber optic cable).

In one aspect, the systems and methods described herein provide for an underwater vehicle to establish an underwater optical communication link between a first cabled observatory 504b and a second cabled observatory 504c. The underwater vehicle carrying an optical communications system according to the present invention may include two optical modems, mechanically coupled by a tether. Each optical modem may include a transmitter having at least one optical source capable of emitting electromagnetic radiation of wavelength in the optical spectrum between about 300 nm to about 800 nm, and a diffuser capable of diffusing the electromagnetic radiation and disposed in a position surrounding a portion of the at least one source for diffusing the electromagnetic radiation in a plurality of directions. In some embodiments, the tether includes a fiber optic cable, copper cable, or any other suitable type of cable. In some embodiments, each optical modem includes at least two optical sources. A first optical source may be configured to emit electromagnetic radiation at a wavelength different from a second optical source.

The first and second cabled ocean observatories may be submerged under a water body at a desired depth, resting on a sea floor or suspended in the body of water. As referred to herein, the terms “cabled ocean observatory” and “cabled observatory” may be used interchangeably. The cabled ocean observatory may be designed around either a surface buoy or a submarine fiber optic/power cable connecting one or more seafloor nodes. In some embodiments, an underwater observatory maybe a stand-alone unit that is not connected to another communication unit by a tether or a cable. The stand-alone underwater observatory may include an independent power source such as a battery to operate independently. As referred to herein, the term “seafloor node” may refer to an underwater communication unit that includes an optical modem or any other suitable communication device. The observatory may also include sensors and optical imaging systems to measure and record ocean phenomena. A cabled observatory may be connected to a surface buoy, one or more seafloor nodes by a cable, a surface ship, or a station on land. In some embodiments, the cable includes a tether as described in further detail below. The cabled observatory may include an optical modem, which will be described in further detail below in reference to FIG. 5. In some embodiments, the optical modem is oriented with a hemispherical diffuser downwards. It should be understood that in some embodiments, the optical modem may be oriented upwards, sideways, or any other suitable direction. To avoid cross-talk among the plurality of modems, different collision avoidance protocols may be used, including TDMA, CDMA, FDMA, SDMA, or any other suitable protocol as described above, as well as entraining secondary emissions according to the present invention. In addition, each modem may communicate on a plurality of optical channels, such as a different wavelength of electromagnetic radiation.

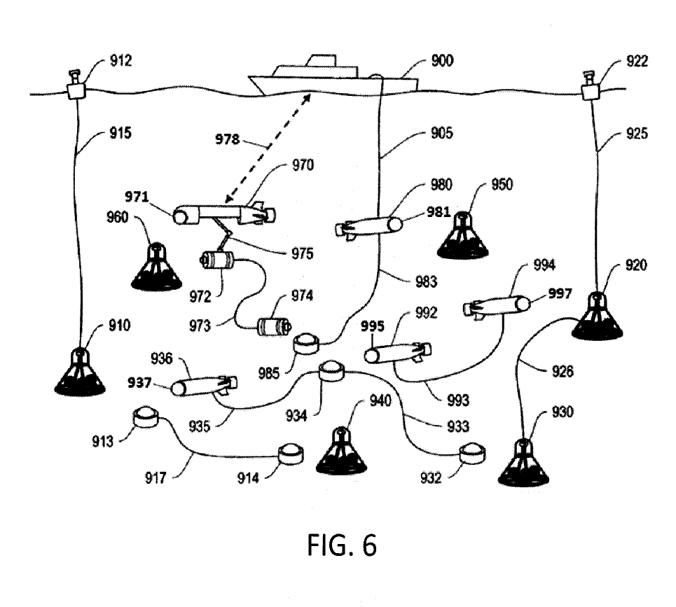

FIG. 6 illustrates an underwater optical communication network including a plurality of underwater optical modems, typically associated with underwater observatories, and underwater vehicles according to an embodiment of the invention. A plurality of underwater observatories 910, 920, 930, 940, 950 and 960, a plurality of stand-alone underwater optical modems 913, 914, 932, 934, 974, and 972, and a plurality of underwater vehicles 936, 970, 980, 992, 994 with secondary emission sources 937, 971, 981, 995 and 997, respectively. Also illustrated are various tethers 917, 933, 935, 973, 983, and 993 that mechanically couple various optical modems. Cables 905, 915, 925, and 926 are illustrated that may connect underwater observatories to one or more surface buoys 912 and 922, underwater observatories to other underwater observatories, or an underwater vehicle to a surface vessel 900. Additional communication techniques can be utilized such as acoustic transmissions 978 between underwater vehicle 970 and surface vessel 900.

Various configurations of underwater observatories and communication networks according to the present invention are depicted in FIG. 6. In a first configuration, a cabled underwater observatory 910 is connected via cable 915 to a surface buoy 912, which resides at the surface of the water. In a second configuration, a cabled underwater observatory 920 is connected via cable 925 to a surface buoy 912, which resides at the surface of the water. Cabled observatory 920 is connected via cable 926 to an underwater observatory 930. In a third embodiment, an underwater observatory may be a stand-alone unit, as illustrated by underwater observatory 940, 950 and 960.

An optical communication network may be established between the plurality of underwater observatories. Stand-alone underwater optical modem 913 may be disposed within an optical range of underwater observatory 910, and stand-alone underwater optical modem 914 may be disposed within an optical range of underwater observatory 940. A tether 917 may mechanically couple underwater optical modem 913 to underwater optical modem 914. Underwater optical modem 913 and underwater optical modem 914 may be deployed using a UUV as described above in reference to FIG. 2.

The network may be extended to include a plurality of nodes. As referred to herein, the term “node” may be defined as an underwater optical modem or a communication unit that is part of a communication network or system (e.g., an optical communication network, an acoustic communication system, or a multi-modal communication system). Underwater optical modem 932 may be deployed by a UUV 936 within an optical range of underwater observatory 930. Underwater optical modem 934 may also be deployed by UUV 936 at a location different from underwater optical modem 932 to facilitate connection to other underwater optical communication links. Underwater optical modem 934 may be mechanically coupled to underwater optical modem 932 by tether 933 and to UUV 936 by tether 935. UUV 936 may include an integrated optical modem that enables it to communicate with nodes in the optical communication network. For example, UUV 936 may navigate to a location within an optical range of underwater optical modem 913, and establish an optical connection with underwater optical modem 913, thereby establishing an optical communication link between underwater observatories 910, 920, 930, and 940.

Faults in the underwater optical communication network may be repaired by reconfiguring nodes in the network. For example, a fault may be detected in tether 926, breaking the optical communication link between underwater observatory 920 and underwater observatory 930. To re-establishing an optical communication link between underwater observatory 920 and underwater observatory 930, optical modems may be deployed at nodes in the network that are connected to the underwater observatory 920 and underwater observatory 930. For example, UUV 994 and UUV 992 may each include an integrated optical modem that may be mechanically coupled to each other by tether 993. UUV 994 may navigate to and establish an optical connection with underwater observatory 920, and UUV 992 may navigate to and establish an optical connection with underwater optical modem 934. An optical communication link may be formed between underwater observatory 930 and underwater observatory 920 through UUV 992 and UUV 994. In some embodiments, each of UUV 992 and UUV 994 is configured to deploy an optical modem (not shown), that is mechanically coupled by a tether to an integrated optical modem. For example, UUV 992 may be configured to deploy a first optical modem that is mechanically coupled by a tether to an optical modem integrated with UUV 992, which is also mechanically coupled to the integrated optical modem of UUV 994 by a tether 993. In some embodiments, the UUV 994 is configured to deploy a second optical modem that is mechanically coupled by a tether to the integrated optical modem of UUV 994, and also mechanically coupled to the integrated optical modem of UUV 992, and the first optical modem that is deployable from UUV 992.

In some embodiments, optical connections may be formed to stand-alone underwater observatories. For example, UUV 980 may deploy underwater optical modem 985 within an optical range of underwater optical modem 934. UUV 980 may include an integrated optical modem and navigate to stand-alone underwater observatory 950. The integrated optical modem of UUV 980 may be mechanically coupled to underwater observatory 985 by tether 983. UUV may be connected to a surface ship 900 by a cable 905. The cable 905 may enable remote control of underwater vehicle 980.

In some embodiments, optical connections may be formed by deploying a set of stand-alone optical modems. For example, UUV 970 may deploy underwater optical modem 974 within an optical range of 985, and deploy underwater optical modem 972 within an optical range of stand-alone underwater observatory 960. In one construction, underwater optical modem 972 and underwater optical modem 974 are connected by physical tether 973.

As further illustrated in FIG. 6, a plurality of different nodes may connected in a linear or a non-linear arrangement. As referred to herein, the term “linear arrangement” may refer to a series of optical modems that may be connected in a non-branching chain. For example, the series of underwater optical modems 914, 913, 936, 934 and 932 may be considered a linear arrangement. As referred to herein, the term “non-linear” arrangement may refer to an arrangement of optical modems or communication units that include branches. For example, the collection of underwater optical modems 972, 974, 980, 985, 934 and 932 may form a branched arrangement that extend from underwater optical modems 934, 974 and 985 as a nexus.

This invention may also be expressed as a system and method for reducing fouling of a surface (e.g., a UV-transmissive surface, a curved surface, and/or a transparent surface) or an element, particularly a optically transparent surface, a UV-transparent material, a window (e.g., a camera window), a lens, a light, a sensor, or a surface unsuitable for an antifouling coating or paint as known in the art, subjected to an aquatic environment (e.g., marine environment, body of water, salt water, fresh water), including providing a plurality (e.g., one or more, two or more, three or more, or at least four) of mounts disposed about or proximate to the surface and extending into the marine environment, each mount housing an LED or other suitable light-emitting source for emitting UV-C radiation from a distal end of each mount, and each mount angling its distal end inward and downward (e.g., positioned to emit light downward) toward and proximate to the surface of the optically transparent element. Each LED is driven to emit UV-C radiation, and emitted UV-C radiation is directed toward the surface of the optically transparent surface or element.

In some constructions, operation of each LED or other light source is coordinated as a “secondary emission” with primary transmission signals as described above for optical communication systems according to the present invention. In other constructions, at least some of the LEDs are turned off during transmission or reception of optical communication signals.

FIG. 7A depicts an optical modem (or transmitter assembly) 1100 with a system 1101 for reducing fouling of a surface of an optically transparent element 1102 in a marine environment. The outer surface of the optically transparent element may be in contact with a fluid (e.g., a marine fluid including water having a measurable salinity, fresh water, salt water, a body of water), making this surface particularly vulnerable to developing biofilm that supports larger organism bio-fouling. The system 1101 may be configured to remove/prevent the formation of biofilm. The optically transparent element 1102 allows for the transmission of light therethrough, enabling communications and sensors reliant on optics to operate within the interior of the optical modem 100, but which can be obstructed through the formation of biofilm and related organisms. Embodiments of this invention are suitable for use with various systems and methods of optically communicating underwater, including those described above and in U.S. Pat. No. 7,953,326, which is hereby incorporated herein in its entirety.

The surface to be protected from biofilms, such as the surface of optically transparent element 1102, is located in some constructions on an end cap 1104 of the node such as optical modem 1100. The optically transparent element 1102 can take many different forms, including a window, a lens (e.g., flat or curved), a sensor, a UV transparent material, among other suitable forms as known in the art. The end cap 1104 may include one or more mounts 1106 extending from an upper side thereof. These mounts 1106 may be disposed near the periphery of the end cap 1104, as depicted in FIG. 7B. The mounts 1106 may be adapted to house or otherwise carry an ultraviolet (UV, including UV-C) light-emitting diode (LED) 1108 at a distal end thereof, such as within a watertight enclosure such as an impermeable housing to protect the LEDs 1108 from the surrounding marine fluid. These LEDs 1108 may provide light in a variety of wavelengths, including wavelengths from about 265 nm to about 295 nm, though greater and lesser wavelengths may be produced, as well (e.g., 200 nm to 220 nm, 200 nm to 240 nm, 200 nm to 280 nm, 240 nm to 265 nm, 240 nm to 295 nm, 270 nm to 295 nm, 280 nm to 300 nm). The enclosure may have a UV transparent port so that UV light from the LEDs 1108 may pass through the enclosure to the optically transparent element 1102.

In some constructions, the mounts 1106 are configured to direct emitted UV-C radiation from the LEDs 1108 toward the optically transparent element 1102, for example, by angling the distal end of the mounts 1106 with the LEDs 1108 inward and downward toward the optically transparent element 1102 or at any suitable angle to irradiate the surface with UV light. In one construction as illustrated in FIGS. 7A-7C, the mounts 1106 extend on cylindrical tubing away from optically transparent element 1102 and then bend toward the optically transparent element 1102. The mounts 1106 may be bent in a fixed position or may be adjustable (e.g., flexible) to provide the more efficient angle for irradiating the surface. Each of the LEDs 1108 is directed toward a different portion of the optically transparent element 1102 in this construction. In some constructions, more than one LED is directed toward the same portion of the element 1102. With the mounts 1106 and the LEDs 1108 on the exterior of the optically transparent element 1102 (i.e., in the marine fluid), they are proximate to the surface to be irradiated at a distance, in various constructions, of approximately 0.5 cm, 1 cm, 1.2 cm, 1.5 cm, 1.7 cm, 2 cm, 2.5 cm, 3 cm, 4 cm, 5 cm, 6 to 8 cm, or less than 8 cm, 10 cm, 15 cm, 20 cm, up to 30 cm, or greater than 30 cm. The LEDs 1108 may be used alone or in conjunction with others, as described below.

In certain constructions, a plurality (e.g., one or more, two or more, three or more, at least four) of mounts comprising LEDs 1110 for emitting UV radiation are mounted on an interior of the optically transparent element 1102 within a watertight housing, proximate to the surface (e.g., less or about 0.1 cm, 0.2 cm, 0.3 cm, 0.4 cm, 0.5 cm, up to 1 cm or more) to be irradiated, requiring any light intended to reach the surface to first pass through the material of the surface 1102. For such constructions, the surface 1102 preferably is made of a UV transparent material (e.g., UV-transmissive) to allow UV radiation to reach the surface. The interior LEDs 1110 may be used alone or in conjunction with the exterior LEDs 1108. The UV radiation is transmitted though the surface to reduce fouling of the surface subjected to the aquatic environment including an aquatic fluid while the surface is in contact with the aquatic fluid.

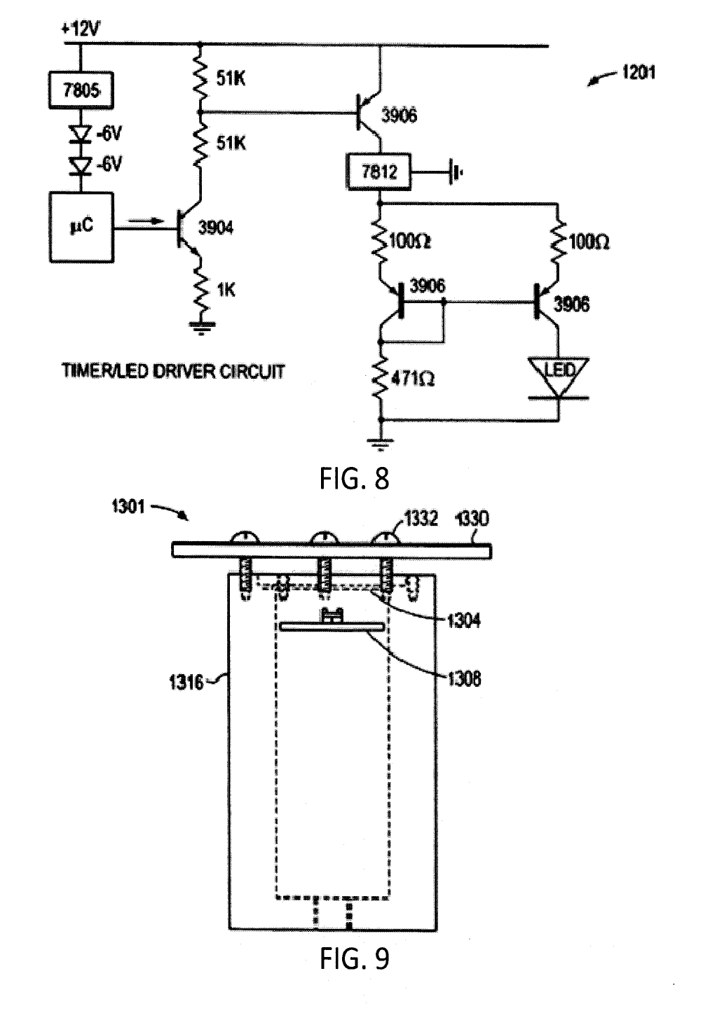

The LEDs 1108, 1110 may be controlled by a timer/driver circuit 1201, as depicted in FIG. 8, or other control circuitry. The control circuit 1201 generally drives the LEDs 1108 and may control the duty cycle of the LEDs 1108, allowing a user to control the period of time the LEDs 1108, 1110 are on (and thus when they are off). The circuit 1201 may maintain a constant duty cycle of the LEDs 1108, 1110 for a period of time, e.g., 80 minutes on and 12 hours off. The duty cycle may be set to any period of time, including at least about 10% of on time compared to total time, and in some instances, about 1%, 2%, 5%, 15%, 20%, 25%, 30%, 40%, 50%, 60%, 70%, 80%, or up to 100% of on time. The system 1101 may be configured to dose the surface with a predetermined amount of light energy and density (e.g., about 0.5 kJ/m², 1 kJ/m² or more) and/or to achieve a desired kill efficiency (e.g., at least about 80%, 85%, 90%, 95%, 97% or 98%, depending on the construction chosen).

A light emitting array 1112 may be used to communicate with another optical device. In some embodiments, the array may be a receiver instead of, or in addition to, being an emitter, and may replace the light emitting array 1112 referred to throughout the specification. The various embodiments of the array may be used for transmitting or receiving optical signals. The electronics controlling the LEDs 1108, 1110 and/or the electronics controlling the light emitting array 1112 may be located on a mounting flange 1114 extending from a lower side of the end cap 1104. The mounting flange 1114 may be protected from the exterior environment by a housing 1116 and an additional end cap 1118. Each of the end caps 1104 and 1118 may have a bore 1120 and 1122 respectively formed therethrough to provide passage into the optical modem 1100, such as for electrical wiring, as depicted in FIG. 7C, and/or for mechanical connections to an object such as a communication node, an observatory, a transducer, an optical modem, an vehicle, an AUV, an ROV, an UUV, a winch, a dock, and a profiler. If necessary, these bores 1120, 1122 may be covered or sealed to preclude introduction of marine fluid into the housing 1116.

To use the system 1101, the user may pre-program a control circuit 1201 to drive the LEDs 1108, 1110 to emit UV radiation. This may be done on a set schedule, as part of a constant duty cycle, or on demand. When an appropriate amount and type of UV-C radiation is directed toward the optically transparent element 1102, biofilm formed thereon is reduced, removed, or otherwise preventing from developing on the surface.

FIG. 9 depicts an experimental setup 1301 for comparing the effects of two separate wavelengths of deep UV LEDs on the growth of biofilms. The purpose of the experiment was to assess the effectiveness of both 265 nm and 295 nm UV LEDs for the purpose of eliminating the primary biofilm that supports larger, obtrusive biofouling on an underwater substrate or window. This experiment was intended to test LEDs as sources of deep UV, as well as to determine the threshold dosages required to prevent fouling. Previous tests disclosed that high doses of ˜260 nm UV emitted from lamps would keep a substrate sufficiently clear. LEDs are of particular interest due to their efficiency, long lifetime (when driven properly), and compact size.

The experimental setup 1301 includes an LED 1308 (one 265 nm LED and one 295 nm LED in separate assemblies), a housing 1316 with a window 1304 for the LED 1308 to project through, and a substrate 1330 mounted to the housing with connectors 1332. Also included, but not depicted, are a timer circuit, a current driver circuit, a power supply, underwater cable connectors, Subconn MCIL2M connectors, general radio connectors, and 5″×8″ enclosures. Substrate 1330 represents substrates 330a and 330b as shown and described relative to FIGS. 4A-4E of U.S. application Ser. No. 13/940,814 filed 12 Jul. 2013, US Patent Publication No. 2014/0078584, which are photographs of substrates subjected to different UV wavelengths over a period of time. These FIGS. 4A-4E and accompanying description, as a portion of the entire contents of the above-mentioned application, are incorporated herein by reference. In each of FIGS. 4A-4E, the substrate 330a exposed to 265 nm is on the left and the substrate 330b exposed to 295 nm is on the right. FIG. 4A is a photograph taken on day 1 of the experiment, FIG. 4B on day 6, FIG. 4C on day 19, FIG. 4D on day 22, and FIG. 4E on day 33 (the final day).

The common timer circuit was programmed to a predetermined duty cycle (i.e., 80 minutes on, 12 hours off). The housings 1316, one containing a 265 nm LED and the other a 295 nm LED (both with individual driver circuits), were sealed by screwing on their respective Lexan™ substrates 330a, 330b (SABIC Innovative Plastics; Pittsfield, Mass.). The housings 1316 were then connected to their respective cables, and dangled underwater approximately 1 m below the low-tide line for optimal sunlight and constant submersion. The cables were then connected to the LED timer circuit, powered by a 12V DC power supply. The date and time were noted, and the substrates 330a, 330b were left to be fouled. Every few days, the housings 1316 were recovered and the substrates 330a, 330b were removed without disturbing any potential growth. The underside of each substrate 330a, 330b was then studied for signs of growth and photographed (see FIGS. 4A-4E). The substrates 330a, 330b were reinstalled and the housings 1316 were again submerged. This process was repeated until the amount of accumulated biofouling indicated that the current duty cycle was less or more than adequate, ordinarily a period of four weeks.

The second test configuration, with a duty cycle doubled to 40 min on and 12 hr off (5%), yielded interesting results. While the substrate 330a radiated with 265 nm UV showed little improvement with the doubling of dosages, the more powerful yet less effective 295 nm LED 1308 was much more successful. A slight biofilm did form on the 295 nm substrate 330b within its irradiated radius, but it was clearly more effective than the 265 nm, lower-power LED 1308. Neither window 1304 supported any kind of growth.

A third test configuration, as indicated in Table 1 below, was configured with a duty cycle of 80 min on and 12 hr off (10%). This time, both substrates 330a, 330b were kept completely clear of fouling, and there was no discernible difference between the effects of the two wavelengths of LEDs 1308.

TABLE 1
LED Configuration for Dataset #3

| | | | | | Worst-Case |
|---|---|---|---|---|---|
| | | | | | Attenuated |
| λ | Kill | Po | | Dosage | Dosage |
| (nm) | Efficiency | (μW) | Duty Cycle | (kJ/m2)* | (kJ/m2) |
| 265 | 95% | 300 | 80 min ON | 1.37 | 0.49 |
| | | | 12 hr OFF | | |
| 295 | 25% | 500 | 80 min ON | 2.29 | 0.82 |
| | | | 12 hr OFF | | |

*Research suggests that 0.5 kJ/m² will eliminate 98% of microorganisms.

Based on the results of this experiment, one 295 nm UV LED 1308 appears to perform just as well or better than a 265 nm UV LED 1308 on the same duty cycle, and is therefore more cost effective, as 265 nm LEDs 1308 typically cost more than 295 nm LEDs 1308 (e.g., $229 for 265 nm, $149 for 295 nm). Dosages of 265 nm UV for antifouling may start at 1.37 kJ/m², and for 295 nm UV may start at 2.29 KJ/m². These dosages may provide a starting point which a user may back off to a threshold dosage, or may be increased by a user to provide a safety factor in irradiation.

To properly ensure transmission of shortwave UV, a specialty UV transparent window 1304 may be used. For wavelengths in the 250-300 nm range, quartz and fused-silica may be suitable material choices. If an internal cleaning system is desired to prevent fouling on a window 1304, the window should be designed for such an application to ensure UV reaches the surface at risk of biofouling. Alternatively, the antifouling system may be external and self-contained. Consideration may also be given to the fact shortwave UV may be subject to high attenuation losses in typical ocean waters, which somewhat limits the distances from the LED to its target substrate for which the LED can be effective.

For this experiment, the shortest possible path length (approximately 1.7 cm) of UV through water was chosen to minimize attenuation losses. While the attenuation coefficients for this range of UV in the waters at the test location were not known, a worst-case scenario estimate with a theoretical coefficient of 0.36 showed that the attenuated dosage to the 265 nm substrate would have been 0.49 kJ/m² for the 80 min duty cycle. This may explain why the lower-duty cycles did not appear to be effective; the dosage required to kill 98% of microbes is 0.5 kJ/m². However, in a different environment, the lower-duty cycles may be sufficient.

The experiment results suggest that both 265 nm and 295 nm UV LEDs 1308 may be effective for antifouling purposes. As 295 nm LEDs tend to be less expensive and equally effective, they may be a preferred choice for the tested duty cycle. It is expected that experimentation with different wavelengths may produce different results. For example, a threshold dosage determined by reducing the UV dosage until one wavelength outperforms the other may be tested at different frequencies to develop a more versatile system that administers less obtrusive, seconds-long dosages at a higher rate. A decrease in off time would allow for lower dosages, decreasing the time for biofilms to accumulate between doses.

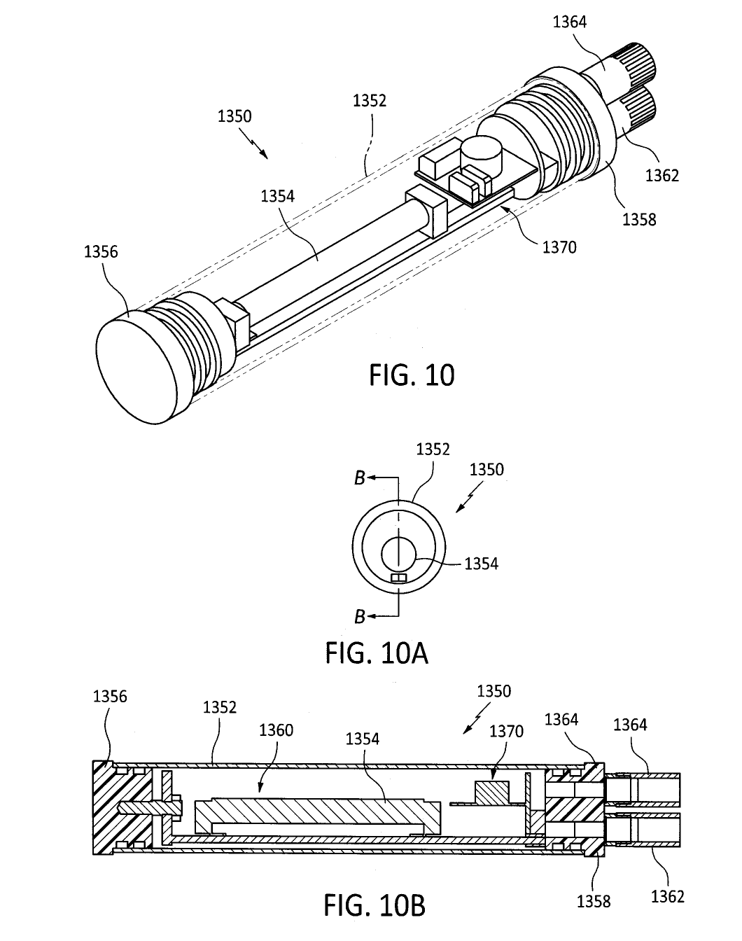

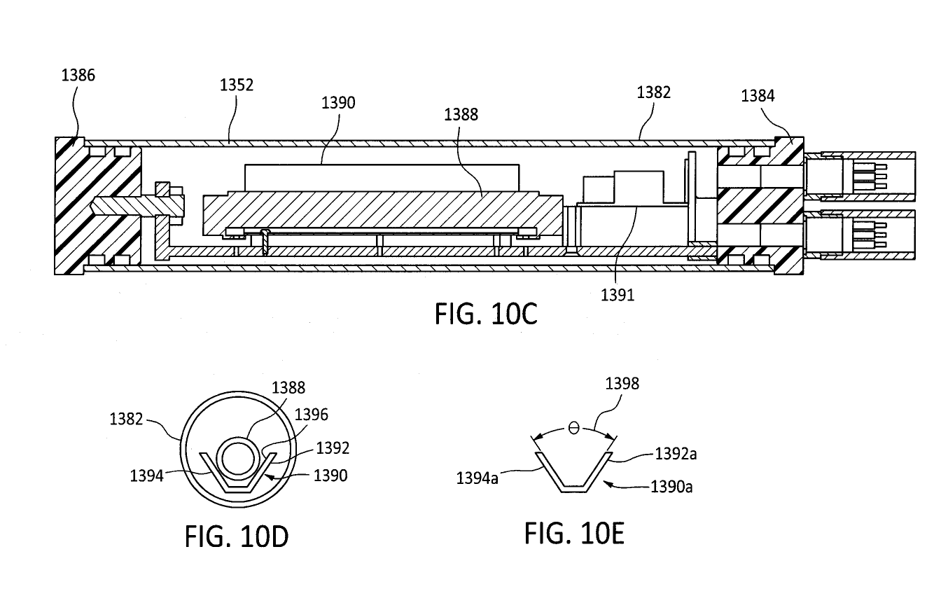

Another construction according to the present invention to reduce biofilms on a surface (e.g., an optically transparent surface, a UV transparent material, a window, a camera window, a sensor, a node, an observatory, a cable, a vehicle, an optical window, a lens, a light, a winch, a spool, a fin, a propeller, among other devices or surfaces exposed to bio-fouling) includes a UV device 1350, FIGS. 10-10B, having a UV-transmissive cylinder 1352, also referred to as a pressure vessel. The cylinder 1352 is formed of silica glass, quartz, fused-silica (e.g., standard laboratory grade fused-silica tubing), glass, plastic, and/or any suitable material known in the art to enable the pressure vessel to operate at depth and to provide the optical window for one or more light sources 1354 for emitting UV radiation toward the surface. In some constructions, certain surfaces are unsuitable to be painted with an antifouling coating as the coating often degrades or chips off, leaving the surface exposed to bio-fouling. In other cases, surfaces such as a transducer (e.g., acoustic or optical) may not operate efficiently when painted and will benefit from alternative forms of antifouling such as the present invention. [000104] End caps 1356 and 1358 cooperate with cylinder 1352 to establish a pressure vessel that retains a gas such as atmospheric air or other gas (e.g., an inert gas, a noble gas, nitrogen gas, combination of gases) within volume 1360 of device 1350. In many embodiments, the pressure vessel 1352 needs not to be pressurized with said gas to withstand the external pressure forces of the surrounding environment, wherein the pressure vessel 1352 is most often adapted to operate up to a depth of 400 m, 500 m, 600 m or more. In certain instances may require pressurization such as when operated at depths greater than 600 m, 800 m, 1,000 m, or more. The end caps 1356 and 1358 may be comprised of any suitable material capable of withstanding the pressure rating of the UV device 1350 such as glass, plastic, or other polymer which is cost-effective and lightweight. One or both of end caps 1356 and 1358 can facilitate external mounting to an object. In one constructions, the UV device 1350 is comprised of glass and plastic materials which reduces corrosion issues in the marine environment and provides a lightweight, economical design.

In this construction, end cap 1358 includes plugs 1362 and 1364 which are adapted to provide mechanical and electrical connections, for power and signal transmission, with an object such as a node, an optical modem, an observatory, a transducer, a vehicle, an autonomous underwater vehicle (AUV), a remotely operated vehicle (ROV), an unmanned underwater vehicle (UUV), the posterior end of a vehicle (e.g., a prop, fins, etc.), a dock, a winch, a profiler, or any suitable object requiring bio-fouling mitigation. In several constructions, the light source 1354 emits UV radiation within the wavelength range of 240 nm and 295 nm, and preferably within a range of 250 nm and 260 nm. In one construction, the light source 1354 is a mercury COTS (Commercial Off-The-Shelf) UVC germicidal fluorescent lamp with an optical output of approximately one watt or more, preferably transmitting 254 nm peak UV-C biocidal wavelength (e.g., 250 nm to 260 nm), drawing approximately 500 mA of at least 12 V operating voltage. In some embodiments, the light source is a single mercury lamp. Using a single light source, compared to multiple light sources, reduces cost and provides an economical device for reducing fouling. In another construction, the light source 1354 is an LED. In some constructions, the UV device 1350 is capable emitting UV radiation (e.g., UV-C) from all angles (e.g., 360 degrees) within the pressure vessel. In other constructions, the UV device 1350 is capable of emitting UV radiation at a specific angle or in a specific direction in a first or normal pattern.

Directed radiation most often employs a configurable (e.g., adjustable) optical reflector or other suitable mirrored surface to enable tailoring of UV emission patterns to particular situations, such as wide-angle versus narrow-angle UV transmission patterns relative to the normal emission pattern of the antifouling light source.

One example of a configurable optical reflector is provided by device 1380, FIGS. 10C-10D, having a cylindrical, optically transmissive tube 1382 and end caps 1384, 1386 that provide a controlled environment for a light source 1388 and electronics 1391. Optical reflector 1390 has parabolic walls 1392 and 1394 with a reflective inner surface 1396. FIG. 10E is a schematic end view of an optical reflector 1390a showing the angle θ, as indicated by arrow 1398, between opposing walls 1392a and 1394a of the reflector 1390a.

In some constructions, the optical reflector is a radiant reflector adapted to provide uniform or substantially even illumination over the surface selected from a metalized plastic surface, a high-polished stainless steel surface, or any suitable material for reflecting UV radiation. In some constructions, the UV devices 1350 and 1380 provide a wide-angle UV emission pattern of at least 50 degrees, 55 degrees, 60 degrees, 65 degrees, 70 degrees, 75 degrees, 90 degrees, 120 degrees, 150 degrees, 180 degrees, and up to 360 degrees. In other constructions, the UV devices 1350 and 1380 use a narrow-angle UV pattern of less than 50 degrees, approximately 45 degrees, 40 degrees, 35 degrees, 30 degrees, 25 degrees, 15 degrees, 10 degrees, or less.

The UV device irradiates the desired surface with UV light at a distance to effectively reduce and/or prevent bio-fouling accumulation. In some constructions, the UV device 1350 is effective at a distance of approximately or at least 0.5 cm, 1 cm, 2 cm, 3 cm, 4 cm, 5 cm, 6 cm, 7 cm, 8 cm, 9 cm, 10 cm, 12 cm, 15 cm, 20 cm, 25 cm, 30 cm, 35 cm, 40 cm, 50 cm, or more depending on environmental conditions.

Device 1350 has an intrinsic operating depth of 600 meters. In some constructions, the pressure vessel 1352 is capable of resisting the external pressure of depths up to 300 m, 500 m, 600 m, 800 m, 1,000 m, 2,000 m, up to 6,000 m, or more.

In one construction, components 1370 comprise control circuitry for driving the light source 1354 and include maintaining a duty cycle (e.g., a constant duty cycle) of the light source 1354 with a programmable control to minimize power consumption. In one construction, the control circuitry comprises a microprocessor and a built-in duty cycle timer to minimize power consumption wherein the duty cycle timer consumes little to no power when no power is provided to the control circuitry. The duty cycle control may be built-in (e.g., internal) to the device 1350 and connected with the light source, providing an in-line, integral timer for emitting UV radiation. In some constructions, the UV device 1350 is pre-programmed with a specified duty cycle; in other constructions, the UV device 1350 is adapted to receive communication from a source (e.g., a sensor, a vessel, a vehicle, a node, etc.) to provide specific programming of the duty cycle. In some constructions, the operation of control circuitry, in particular the duty cycle timer, may be adjusted or set to a specific program from a remote source separate from the device 1350 such as a vessel or other facility by means of a suitable communication connection (e.g., wired data connection, satellite connection) or through a connection established through the object to which the device 1350 is connected. In certain constructions, components 1370 include a power switch such as a mechanical relay, a solid state relay or FET switch.

In some constructions, the inventive antifouling device 1350 is used in conjunction with another means for deterring bio-fouling such as an antifouling coating. Any suitable antifouling coating may be used as determined by one of ordinary skill in the art including a zinc-based antifouling paint (ePaint®), a non-stick paint (ClearSignal®), and a copper-based paint. In such cases, the duty cycle of the system and the dose of UV light may be reduced to a minimum to efficiently reduce bio-fouling and minimize power consumption.

It is also within the scope of the present invention to form a complimentary multi-modal communication system that incorporates both the long range of acoustics and the high bandwidth of optics for use in an amorphous medium (e.g., a body of water, fluid, salt water, fresh water, atmosphere, surface boundary). In some constructions, a high-functioning, multi-modal communication system provides new capability to the diver for various applications including clandestine underwater operation wherein the multi-modal (e.g., bi-modal) system provides one or more means of communication including, but not limited to, optical communication, acoustic communication, radio frequency, and a combination thereof. The combination of optical and acoustic technologies, along with developments specific to the diver application, can provide data, text, voice, video (e.g., full motion video), and voice push-to-talk (PTT) to divers and other nodes disposed in a body of water (e.g., underwater), above water, on shore, and through the water-atmosphere surface boundary (e.g., the surface boundary).

Voice PTT is facilitated by components adapted to switch from voice reception mode to transmit mode for full duplex communication (i.e., both nodes can communicate with each other simultaneously) such as the Wave Relay Radio Man Portable Unit Gen 4 (MPU4 available from Persistent Systems, LLC). Divers utilizing PTT may also use a positive-locking wet-mate-able connector capable of being manipulated with dive gloves without the need for an external translator (e.g., plug in a headset directly). The system may be configured by the operator to allow audio communications to be uninterrupted (e.g., first or current transmission cannot be interrupted) or interruptible (e.g., most current transmission always interrupts).

In some constructions, a multi-modal communication system according to the present invention is generally capable of operating in an amorphous medium to broadcast a signal through one or more mediums to a node capable of detecting the signal. The system comprises a multi-modal primary node capable of producing a primary signal and transmitting the primary signal and at least one multi-modal secondary node, preferably a plurality of nodes (e.g., multiple secondary nodes), separate from the primary node capable of detecting the primary signal and optionally producing a secondary signal. Communication between the nodes may be provided through optical communication (e.g., optical link), acoustic communication (e.g., acoustic link), radio frequency, and a combination, as determined by the desired operation.

In many constructions, a system according to the present invention utilizes optical communication particularly for high speed communication and acoustic communication for long range communication. Furthermore, the amorphous medium may be considered by the system and/or the user in selecting the suitable mode of communication. Optical communication may be employed underwater and above-water, connecting a plurality of nodes from underwater, on the water surface, the atmosphere, and from shore. Acoustic communication generally employed underwater often as a backup mode of communication for instances when optical communication is not optimal (e.g., excess communication range, physical damage to optical communication network, high levels of turbidity, scattering, absorption, among other unfavourable environmental conditions). In particular, turbidity causes light attenuation by absorption and scattering. In highly turbid water, multiple scattering and absorption will dominate the channel and cause a reduction in range compared with less turbid water. Scattering is mainly dependent on the size and composition of particles suspended in water and can be caused by many things including sediments and phytoplankton. Scattering is very wavelength-dependent, and the scattering of 385 nm light can be significantly worse than that of 450 nm light, which is worse than that of 700 nm light. In coastal waters, green light travels farther than blue largely because of scattering effects. In most constructions, the optical link should be able to function under all light conditions and provide enough bandwidth to transfer video and PTT traffic while rarely becoming completely inoperable. In the case of poor optical conditions, a diver may connect a surface buoy or other node via acoustic communication and download video or data.

Each of the primary and the secondary node(s) may be any suitable communication unit capable of providing a signal through an amorphous medium, but are most often selected from a diver node, a buoy node, an underwater buoy node, an observatory, a UUV, a UAS (Unmanned Aerial System), a unmanned aerial vehicle (UAV) such as a drone, an aircraft, a surface vessel, an off-shore platform, and a shore-based node. The present invention also envisions that other vehicles, apparatus, and devices may be incorporated with the system as deemed suitable by one of ordinary skill in the art. In general, the multi-modal system provides communication using nodes capable of broadcasting a signal a distance of at least or approximately 0.5 m, 1 m, 5 m, 10 m, 15 m, 20 m, 30 m, 40 m, 50 m, 100 m, 200 m, 500 m, 1,000 m, up to 6,000 m, or up to full-ocean depth.

In some constructions, the optical link operates in the wavelength range of about 300 nm to 800 nm, preferably between 300 nm to 400 nm, and most preferably at approximately 380 nm to 385 nm and includes a photomultiplier tube (PMT) as the primary optical detector, coupled with a photodiode for use in high ambient light conditions which are generally caused by the sunlight, diver-held lights, or vehicle lights. When using a photodiode receiver, ambient light primarily adds noise to the input signal. In the case of PMTs, ambient light can quickly exceed the maximum amount of light the tube can safely receive, which effectively reduces the sensitivity of the receiver. Thus, large photodiodes are much less sensitive and handle ambient light better than PMTs, but they are limited in speed, which affects range. Longer wavelength ranges can be expected with better environmental conditions (e.g., water clarity, dark). The system can operate at data rates of at least 1 megabit per second (Mbps), preferably 5 to 20 Mbps or about 10 Mbps, as desired.

For clandestine operations requiring passive stealth and non-visible communications that are not easily detectable by other parties, communication wavelengths outside of the visible spectrum are desirable, that is, communication wavelengths either (i) less than 400 nm or equal to about 385 nm or (ii) equal or greater than 700 nm. In such cases, near-infrared (NIR) LEDs are used for wavelength ranges of about 720 nm and 740 nm, and near-ultraviolet (NUV) LEDs are used for ranges of about 380 nm to 390 nm. A comparison of the various wavelength ranges depicting their benefits and drawbacks is shown in Table 2.

TABLE 2
Table 2. Wavelength Concerns for Optical Communication

| Wavelength | Benefit | Drawback |
|---|---|---|
| <385 nm | Not easily detected | Highly scattered |
| (NUV) | Clandestine | Some fluorescence possible |
| | COTS source | Potential eye hazard |
| | | Expensive |
| 400 nm to | Best source for clear water | Highly visible |
| 520 nm | Most power efficient source | |
| 520 nm to | Best for coastal waters | Highly visible |
| 600 nm | | |
| >700 nm | Not easily detected | Highly absorbed |
| (NIR) | Clandestine | |
| | Less scattered | |
| | COTS source (inexpensive) | |

It is another aspect of the present system to provide nodes which are operator configurable to employ “hopping” to reduce the probability of interception when used for clandestine operations. Hopping may include frequency hopping (FH) in acoustic communication and wavelength hopping for optical communication as known by one skilled in the art. [000122] Communications according to the present invention can be enhanced by optical filtering by minimizing the amount of ambient light (e.g., daylight, light, wavelengths of approximately 390 nm to 700 nm) received by the PMT which is particularly valuable for communication during daylight. One construction of the optical filtering is further described in Example 1 below.

In some constructions, the acoustic link utilizes a carrier frequency greater than 10 kHz, 20 kHz, 30 kHz, 40 kHz, and preferably greater than 50 kHz and have variable bandwidth to be able to transition between close ranges (less than 100 m) to one km or more.

Data rates depend on the actual conditions of the amorphous medium. Preferably, the system is flexible and adaptable to the environment so that telemetry (text), voice, video, data, and images (e.g., compressed still images) can be sent over the link at the best possible rate.

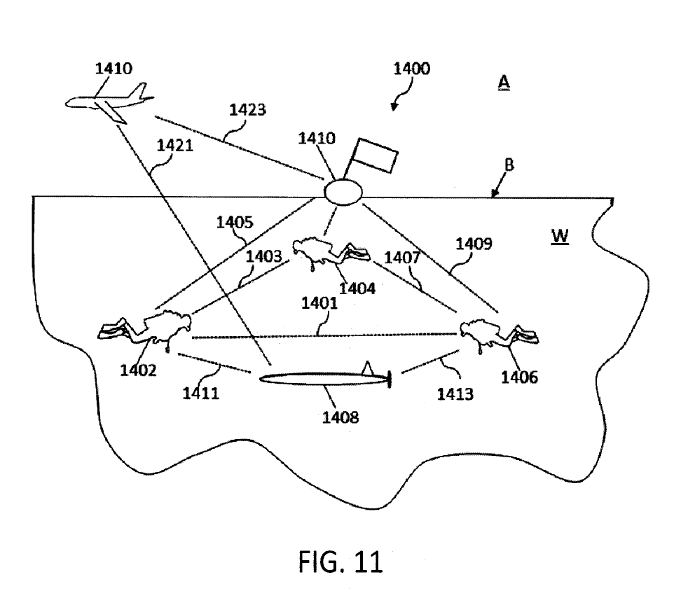

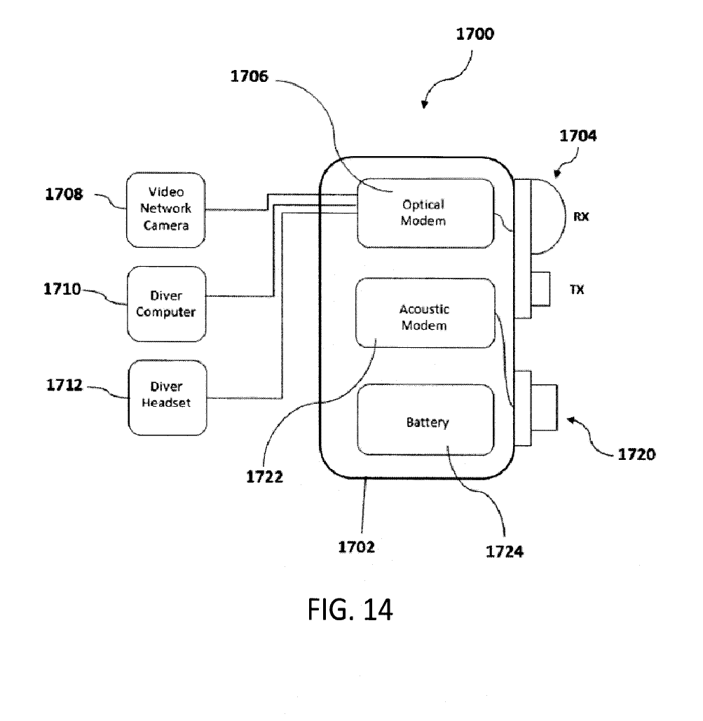

Alternative configurations for communication among a plurality (e.g., multiple) nodes according to the present invention are illustrated in FIGS. 11-13. System 1400, FIG. 11, includes divers 1402, 1404, and 1406 optically communicating underwater with each other via links 1401, 1403, and 1407. One example of a diver node is provided below in FIG. 14, in which an optical system comprising an optical modem is the primary mode of communication when the diver node is in optical communication range with another node, and a secondary acoustic system for back-up communication, such as in high-turbidity, poor-visibility conditions. The system 1400 may support a plurality nodes including divers, underwater vehicles, and surface buoys as well as aerial vehicles.

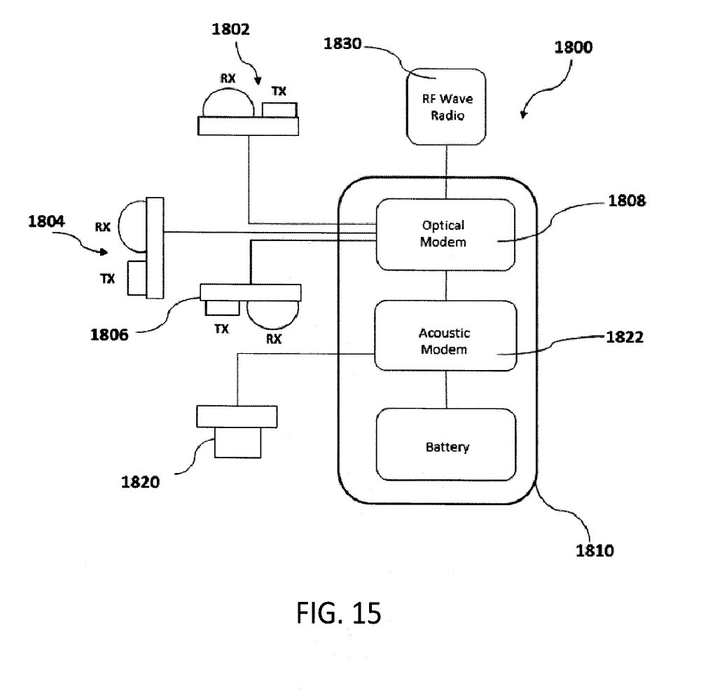

Additional links 1411 and 1413 are shown between divers 1402 and 1408 with an UUV 1408, and with links 1405 and 1409 to a buoy or float 1410, which alternatively is a mobile surface vessel, at boundary B between water W and atmosphere A. The buoy node preferably takes advantage of its larger size to provide more battery power thus enabling the use of multiple optical transceivers as shown below in FIG. 15. In some constructions, using multiple discrete receivers will enable the buoy system to identify the direction of the diver node and only transmit on the LEDs pointed in the appropriate direction. System 1400 includes one or more communication links to an UAV or an aircraft 1420 such as RF link 1421 with UUV 1408 and RF link 1423 with buoy 1410.

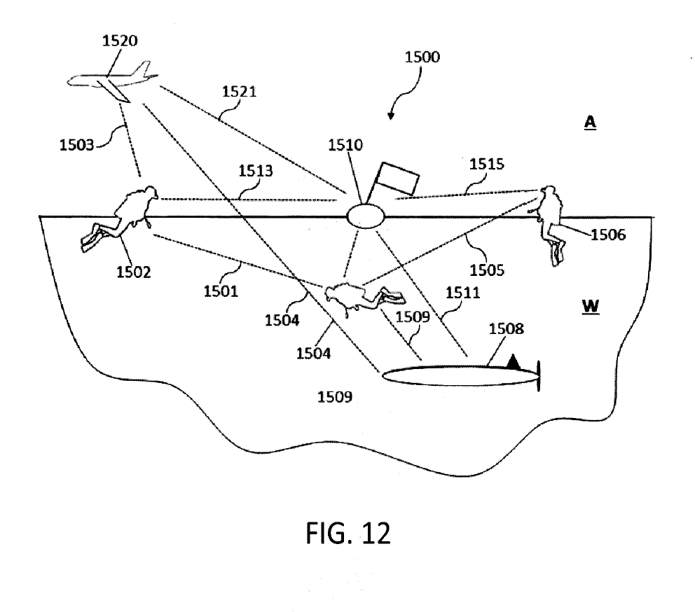

System 1500, FIG. 12, is similar to system 1400, FIG. 11, with some of the divers 1502 and 1506 also communicating above the surface of the water via link 1503 directly to aircraft 1520 as well as indirectly through buoy 1510 via links 1513 and 1515, respectively. Also shown are links 1501 and 1505 with underwater diver 1504, who also communicates via link 1509 with UUV 1508. Communications between UUV 1508, buoy 1510 and aircraft 1520 are show as links 1507, 1511 and 1521.

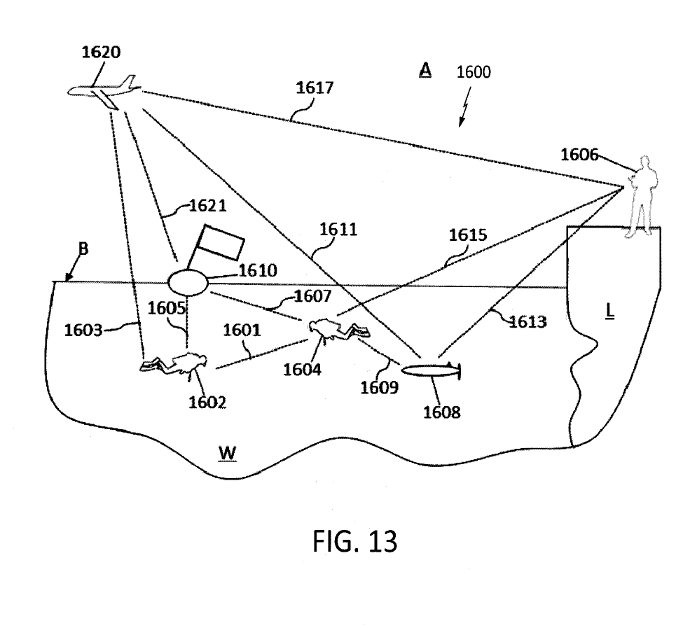

System 1600, FIG. 13, is similar to systems 1400 and 1500 while also including communication with a shore-based node such as a human 1606 (or shore-based node) standing on land L and interacting directly with one or more of a UUV 1608, a diver 1604, and an airplane 1620 via links 1613, 1615 and 1617, respectively. In this construction, diver 1602 communicates directly with diver 1604, a buoy 1610, and an aerial vehicle 1620 via communication links 1601, 1605 and 1603, respectively. Diver 1604 also communicates directly with buoy 1610 and UUV 1608 via links 1607 and 1609, respectively. The UUV 1608 and buoy 1610 also communicate directly with aerial vehicle 1620 via links 1611 and 1621 in this example. In some situations, one or more of the mobile nodes illustrated above in FIGS. 6 and 11-13 is a fish or a marine mammal such as a porpoise or a seal.

As depicted in FIGS. 11-13, the communication system may utilize a plurality of communication modes including optical, acoustic, and RF. In several constructions, optical communication via optical links is primarily used underwater and above water to provide high speed (e.g., high bandwidth) communication among the nodes disposed underwater, on the surface, in the atmosphere (e.g., in the air), or on land. Acoustic communication through acoustic links is generally used underwater among underwater nodes and surface nodes. In some constructions, aerial nodes, such as aircrafts or UAVs, may utilize RF links to communicate with other nodes, particularly nodes in the air, on the water surface, or on land, but may also engage optical links with other nodes when within optical communication range. In other constructions, surface buoy nodes or surface vessels may use optical or acoustic communication to nodes disposed underwater and RF or optical communication to nodes disposed above water or in the atmosphere.

As a general design aspect, preferably each node is capable of transmitting within 60 degrees along at least one plane of orientation and receiving within 360 degrees field of view. In one construction, one or more or all of the nodes is capable of transmitting and receiving at 360 degrees along at least one plane (e.g. horizontal) and is capable of transmitting and receiving along at least 60 degrees, preferably at least 90 degrees, in a transverse plane (e.g. vertical) to achieve at least a hemispherical zone of communication.

Additional aspects of the underwater nodes often include certain physical and operating requirements to maintain the integrity of communication as well as clandestine operation. In several constructions, the nodes disposed underwater are able to communicate at any distance up to 30 m, 40 m, 50 m, 60 m, 70 m, 80 m, 100 m, or more. In some constructions, the underwater node weighs less than 7 lbs, 5 lbs, 3 lbs, and 1 lbs or less in air. The node buoyancy may range ±5 lbs, ±2 lbs, and ±0.1 lbs or less. When employed, the nodes are generally capable of operating at temperature ranges of at least 15° F. to 120° F. in air and 28° F. to 100° F. in water. In the instances of strong shocks or vibrations, the nodes are designed to withstand conditions of shock greater or equal to 4 g, 40 ms, ½ sine, 1,800 pulses minimum and shocker greater or equal to 20 g, 20 ms, ½ sine on 3-axes, 18 pulses minimum. When underwater nodes are brought to the surface and/or out of the water, such nodes are then able to communicate with other nodes above the surface without additional operator intervention.

Diver node 1700, FIG. 14, includes a watertight housing 1702, an optical system comprising an optical transceiver 1704 with receiver RX and transmitter TX connected to an optical modem 1706 that communicates with a video network camera, a diver computer 1710, and a diver headset 1712 in this construction. Housing 1702 also holds an acoustic transducer 1720, an acoustic modem 1722, and a battery 1724. The optical and, to a lesser extent, the acoustic links are primarily line-of-sight systems that must be pointed in the proper direction for optimum functioning. The housing 1702 may be adapted to allow communications and/or operation at depths of at least 1 m, 5 m, 10 m, 20 m, 30 m, 40 m, 50 m, up to 100 m, up to 200 m, up to 500 m, up to 1,000 m, and up to 6,000 m, or more (full-ocean depth).

In one construction, the diver node utilizes a hemispherical receiver specifically, but not limited, for night operations. In another construction, the diver node uses a 90 degree full angle receiver particularly for daytime operations. In another construction, to conserve power, the diver transmitter initially has a limited 90 degree field of view which may be mounted to the diver's head (or other portion of the diver) for aiming purposes. In general, the diver node 1700 is less than 6″ in any one direction with the exception of the acoustic transducer and optical transceiver both which are often exposed to the surrounding environment for optimal performance. However, the diver node 1700 may be designed to any suitable size to meet the communication requirements of the communication system.

Several constructions are envisioned utilizing the multi-modal communication system. Although a primary objective of the constructions described below revolves around video communication, all constructions may also include text, image, and/or data communication in addition or in place of the streamed video. In one construction, one diver streams (e.g., communicates, transmits) video to another diver underwater and additionally may communicate via voice (e.g., PTT) separately in time or simultaneously with the video streaming. In one construction, video is streamed from a UAS (Unmanned Aerial System) via RF or optical link (if range is acceptable) to a diver using a buoy node as an intermediate point of communication; additionally, the diver may stream video to the shore-based node of the UAS using a buoy node as an intermediate point. Furthermore, it is an objective of the present invention to provide video streaming in both directions simultaneously and if desired, with voice communication between the diver and the UAS or the shored-based node of the UAS at the same time.

In other constructions, streaming from a UAS node or other aircraft is streamed to all of the diver nodes in the underwater network via a buoy node simultaneously. Additionally, streaming may be provided from the UAS node to a single diver in the underwater network via a buoy node. Furthermore, a diver may stream to all the other divers and to the shore-based node of the UAS via a buoy node. Voice communication between all of the nodes may take place simultaneously or offset in time using the buoy node.

Furthermore, the constructions employ an underwater vehicle, such as an UUV, capable of RF, optical, and acoustic communication to communicate to other nodes disposed underwater, on shore, or in the air. A UAS may stream to all the divers or a single diver underwater via the UUV. Correspondingly, a diver may stream to one or more or all of the other divers and the shore-based node of the UAS via the UUV. Voice communication may also be provided simultaneously in addition to the streamed communication.

In one construction, divers transition to the surface and stream full motion video or other forms of communication from one diver on the surface to another diver or node on the surface using the same nodes utilized underwater. In another construction, divers transition to the surface and stream full motion video from one diver to all the other divers using the same nodes utilized underwater. In another construction, divers transition to the surface and two divers stream video to each other with at least one other diver receiving at least one of the video streams using the same nodes utilized underwater. In yet another construction, divers transition to the surface and communicate via voice between all divers above the surface using the same nodes utilized underwater. In another construction, simultaneous voice communications occur between all nodes and at least one, preferably two or more, full motion videos are streamed using the same nodes utilized underwater.

Buoy node 1800, FIG. 15, includes an optical system with multiple (e.g., one or more, at least two, three, four, five, six, seven, or eight or more) optical transceivers 1802, 1804 and 1806 in communication with optical modem 1808 within housing 1810. At least one acoustic transducer 1820 communicates with an acoustic modem 1822. Long-range communications are provided through RF Wave Radio 1830.

In one construction, the buoy node 1800 uses a hemispherical transceiver such that all the diver and other nodes are in the field of view most if not all the time. In one construction, buoy node 1800 operates with eight simultaneous connections to additional nodes. In constructions specifically designed for clandestine operations, buoy node 1800 is generally made as small and as natural-looking as possible; such buoy nodes may be designed to be no more than 3,000 inch², 2,000 inch², 1,800 inch², 1,500 inch², 1,200 inch², 1,000 inch² and most preferably 200 inch² or less on the surface. The buoy node 1800 most often is able to hold all of the communication components internally and in some cases, a RF payload (e.g., the components necessary for facilitating RF communication).

Operational aspects of the buoy nodes often include the capability to communicate to any underwater node(s) and to relay RF communications. In some constructions, the buoy node is able to hold all non-RF (e.g., optical, acoustic) components required to communicate with sub-surface nodes, an RF payload, a Wave Relay Radio, and GPS (Global Positioning System) antennas. The buoy node is often capable of relaying underwater data communications to an RF radio via Ethernet and may buffer data communications received from an RF radio and relay them to underwater nodes when the underwater nodes are within communication range. The buffer capacity is often at least 50 Mbytes, 100 Mbytes, 200 Mbytes, 225 Mbytes, 300 Mbytes, 400 Mbytes, or up to 500 Mbytes or more. The buoy node is able to be submerged in water and deployed from a depth up to 50 m, 100 m, or greater than150 m.

Other nodes most often comprise similar systems including at least one or more of an optical transceiver, an optical modem, an acoustic transducer, an acoustic modem, and a power source which may be an individual battery or a connection to another object such as a vehicle. In some constructions, each node will provide a total bandwidth of 5 Mbps during daylight operation and 10 Mbps during night operation. Bandwidth is typically split between receive and transmit channels and can vary based on demand. In one construction, the typical 5 Mbps bandwidth allocates approximately 4.8 Mbps to transmit video and voice and 0.5 Mbps to receive voice.

The multi-modal communication system may comprise a plurality of nodes, and each node may provide point-to-point communications (one-to-one communication), multi-point communications (e.g., more than one node simultaneously communicating with a node, wireless mesh network), and/or may host one or more silent listeners (e.g., 2, 3, 4, 5, up to 8, up to 10 or more, or unlimited). Multiple access operation shares the bandwidth between the host node and the transmitting listeners. In some constructions, the system is capable of operating with at least eight simultaneously connected nodes. Furthermore, at any specific moment, the system may automatically use any node as a primary node to relay communication to other nodes that were not within the original node's field of view or not within the original node's communication range.

In one construction, the optical modem includes a main processor having an Advanced RISC (reduced instruction set computer) Machines (ARM) processor combined with a field-programmable gate array (FPGA). The ARM processor is used for high level functions such as user interface, Ethernet interface, and traffic control and routing over the optical and acoustic links. The FPGA is responsible for all aspects of optical physical layer including modulation, error correction, time-division multiplexing (TDM) framing, and high-speed analog-to-digital conversion (ADC) operation and sensor control. This element also provides the interface for the diver headset and performs the ADC and compression tasks. Temporary data buffering is provided for storing video and voice data that will be relayed at a later date. Typical stream-based protocols for video and voice transmission are not optimized for relaying; this task requires an additional function of recording the stream into a file that can be transferred or played at a later date.

In some constructions, the system is capable of converting transmissions to and from IEEE 802.3 Ethernet format. The Ethernet interface generally comprises autosensing IEEE 802.3 10/100 BASE-T Ethernet capabilities and utilizes an internet protocol compliant with TCP/IP IPv4 and IPv6. In one construction, the system may comprise a static IP address; however, the system may have an integrated DHCP Server and able to utilize a static IP address based on the operator configuration. Additionally, the system may have Built-In-Test Capabilities with a “Fault” indicator and fault messages and status available on the Ethernet interface.

The transmit portion of the optical transceiver includes an emitter composed of a bank of light-emitting diodes (LEDs) and LED driver printed circuit boards (PCBs). The high-power LEDs used in these systems are typically used for lighting and other applications. In order to achieve high data rates, small strings of LEDs are driven by a matching driver circuit and can achieve switching speeds fast enough for data rates up to 20 Mbps or more. The receive portion of the transceiver consists of a PMT for dark, long-range operation and a photodiode for high ambient light conditions. An analog interface PCB provides two high-speed input filters and power control for separate PMT and photodiode inputs. The transmit portion may also comprise a signal meter or link indicator to indicate the receive signal strength available.

The acoustic system comprises an acoustic modem subsystem consists of a fixed point digital signal processor (DSP) such as a Micromodem, a floating-point coprocessor, and analog transmit/receive circuitry on another board. The low-power fixed-point DSP is responsible for acoustic link management and interfacing with external devices including the user data and ADC. The coprocessor implements an adaptive equalizer with built-in error-correction and Doppler estimation and compensation. To save power, the coprocessor is off except when acoustic data is being actively received.

The acoustic transducer generates and receives acoustic signals. Because the transmit and receive functions are shared on one transducer (and will utilize the same band), the acoustic system typically are single-duplex. In some constructions, the transducer frequency is selected after additional analysis and discussion with the end user, and is typically between 50 kHz and 150 kHz.

The power source for the system preferably provides 200-300 watt-hours and is constructed of lithium primary or lithium-ion (Li-ion) batteries. Maximum power consumption of approximately 50 watts will occur when the optical transmitter is in constant transmit mode such as when transmitting video. When waiting for acoustic or optical communications, the system will require approximately 5 watts. The battery represents a significant portion of the weight of the diver node and will be selected according to selected diver mission plans. Commercial rechargeable battery assemblies can be utilized for the diver unit.

Transitions from optical to acoustic connectivity preferably are managed to assign and enforce priority to essential data such as voice. Data pathways and traffic control are managed by the optical modem processor. Traffic control rules are employed for different operational states such as all optical, optical/acoustic, and acoustic only modes.

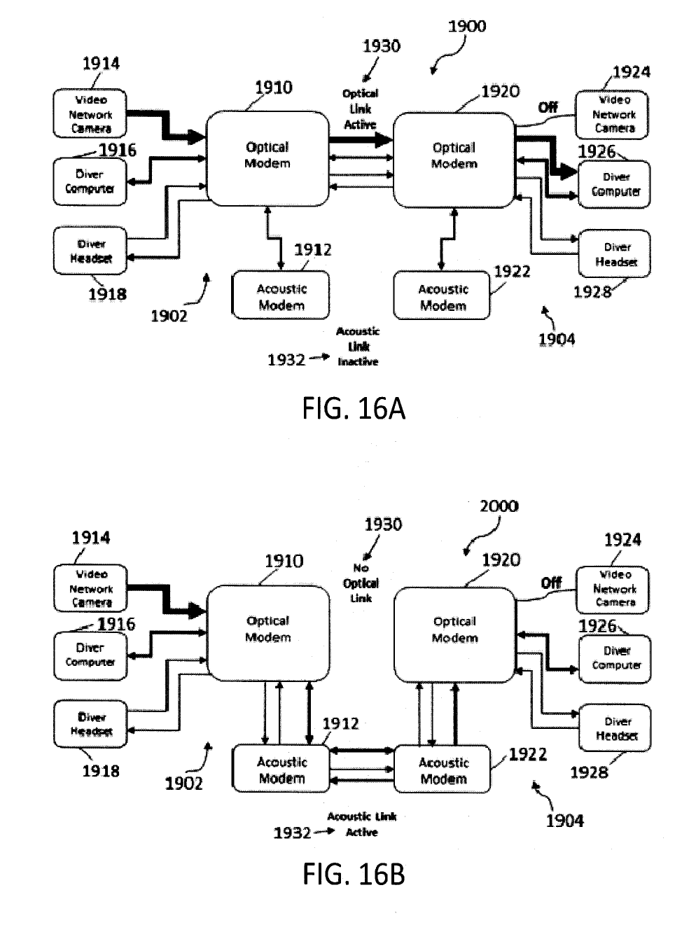

An example of video transfer with bidirectional PTT voice traffic is shown in FIG. 16A for optical transmission mode 1900 between nodes 1902 and 1904, with optical link 1930 active, and in FIG. 16B for acoustic transmission mode 2000 in which acoustic link 1932 is active between nodes 1902 and 1904. In this construction, node 1902 has optical modem 1910, acoustic modem 1912, video network camera 1914, diver computer 1916, and diver headset 1918. Node 1904 has optical modem 1920, acoustic modem 1922, video network camera 1924, diver computer 1926, and diver headset 1928.

When the optical link 1930 is operational during optical mode 1900, FIG. 16A, all traffic is sent over the optical link 1930. If the optical link were dropped and video traffic, such as generated by video network camera 1914 and/or camera 1924, were sent over the acoustic link 1932, the video traffic would exceed the bandwidth of the channel and communications would be lost. Once the link controller identifies that the optical link 1930 has been lost, video transfer is stopped and only PTT and other critical data is sent acoustically during acoustic mode 2000, FIG. 16B. Stream-based video transfer protocols such a H.264 typically require bidirectional communications to start and maintain the transmission of a video stream.

In some constructions, the multi-modal system comprises an Auto Connect/Reconnect mechanism for signal fadeout or dropout. In the instance where the communication link is lost, the system may self-heal and form a new link automatically to continue signal transmission between two or more nodes. In general, the system will reconnect the communication link with 30 sec, 20 sec, 15 sec, 10 sec, 5 sec, and preferably within one second. However, the reconnection time may be primarily driven by the automatic gain control (AGC) and transitions from no light to maximum light may take longer than one second. In a specific construction, the nodes reconnect the communication link automatically within 0.25 seconds of a fadeout or a dropout.

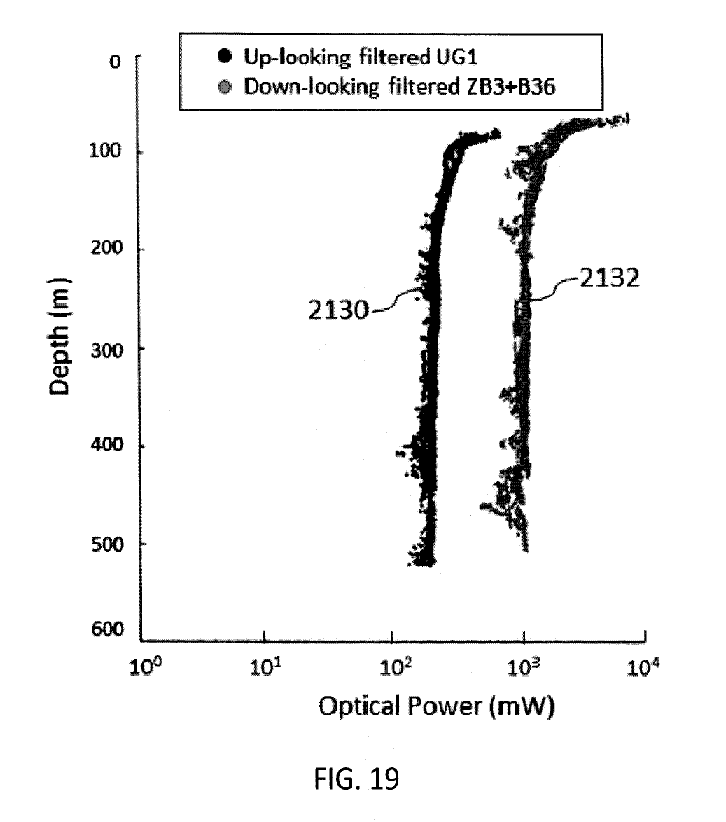

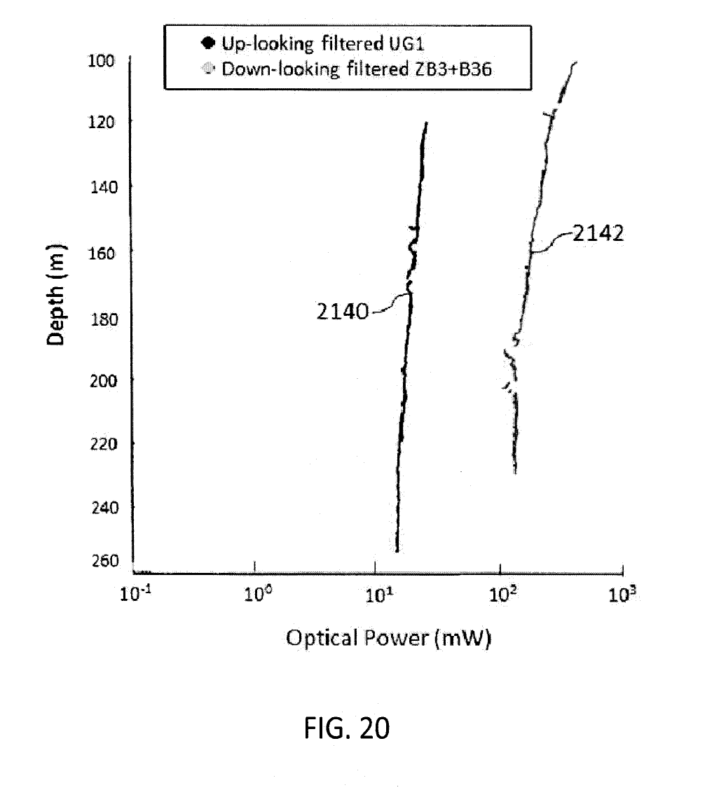

As an Example 1, one construction of the optical filtering applied to daylight optical communications is described as follows. A Conductivity/Temperature/Depth (CTD) rosette device was used on the vessel Atlantis, operated by the Woods Hole Oceanographic Institution (WHOI), as a platform for testing daylight operation of the bi-directional optical modem. An upward-oriented battery-powered optical modem was suspended below the CTD rosette at distances of 15 m and 25 m as described below in relation to FIGS. 19 and 20, respectively. The complimentary downward-oriented optical modem system was mounted on the CTD rosette, with connectivity through the CTD cable.

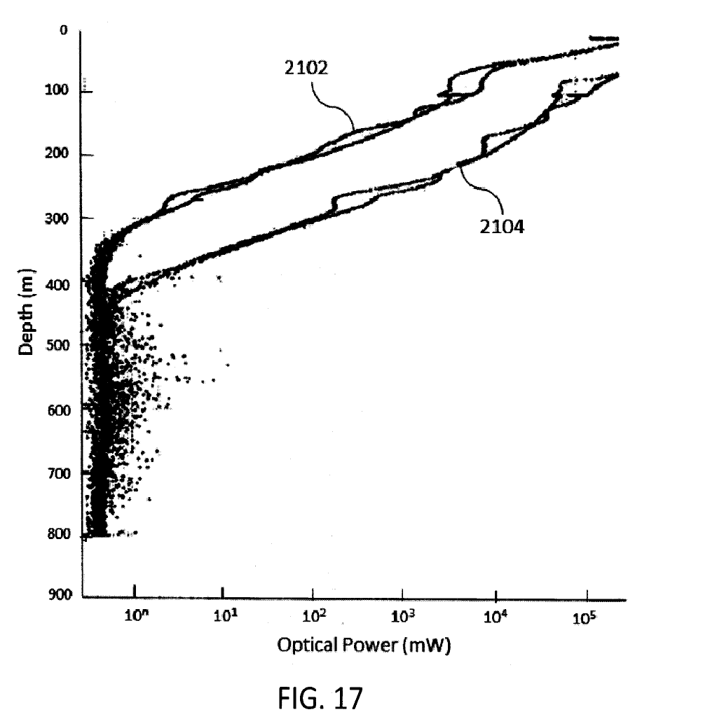

Prior to the communications testing, a pair of optical receivers was deployed on the CTD rosette as power meters: one facing upward and the other facing downward, both without optical filters. As shown in FIG. 17, this data presents solar background as optical power on the x-axis versus depth in meters as the y-axis. The downward-facing receiver, curve 2102, experiences approximately the same power level as the upward-facing receiver, curve 2104, at 100 m more depth, i.e. if the upward-facing receiver were suspended 100 m below the CTD rosette, both receivers would register the same optical power.

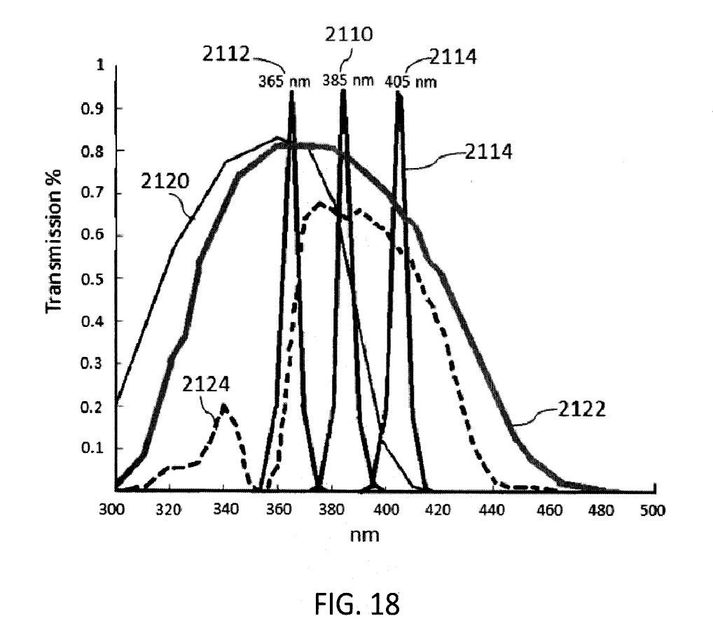

In order to operate in daylight, 385 nm wavelength LED emitters were selected, capable of sustaining a 10 Mbps optical link, combined with absorptive glass optical filters on the receivers, chosen to block most of the visible spectrum (e.g., about 400 nm to 700 nm). Curve 2110, FIG. 18, represents the emitter spectrum for 385 nm wavelength LED, while curves 2112 and 2114 represent emitter spectra for 365 nm and 405 nm LEDs, respectively. Since the upward-facing system is exposed to more solar background, the more aggressive optical filter option (UG1, 2 mm stack), optical filter spectral transmission curve 2120, was employed on the upward-facing receiver. The downward-facing receiver utilized a combination filter (B370+B36, 4 mm stack), spectrum curve 2124. Also illustrated is filter B370 (ZB3, 3 mm stack), transmission spectrum curve 2122.

For the 15 m separation test, an optical link was established at an 80 m CTD depth, where the un-filtered solar background is approximately 10 μW looking down or 100 μW looking up. The optical filtering on the upward-looking receiver, curve 2130, FIG. 19, reduces the optical power at 80 m (background-dominated) from 100 μW to 0.7 μW. The optical filtering on the downward-looking receiver, curve 2132, reduces the optical power at 80 m from 10 μW to 3 μW. Using FIG. 17 as a reference, an optical link without filtering in similar conditions (15 m separation, daylight), would otherwise operate only at depths below 300 m, as limited by upward-looking solar background.

For the 25 m separation test, an optical link was established at a 105 m CTD depth, where the un-filtered solar background is approximately 3 μW looking down or 60 μW looking up. The optical filtering on the upward-looking receiver, curve 2140, FIG. 20, reduces the optical power at 105 m depth from 60 μW to 10 nW. The optical filtering on the downward-looking receiver, curve 2142, reduces the optical power at 105 m depth from 3 μW to 0.1 μW. This background level limits the optical link range, since the signal must overcome 0.1 μW background in order to make a link.

Comparing FIGS. 19 and 20, there is a decrease in received signal power (taken as optical power below 200 m depth) of approximately one decade per 10 m of separation. This puts the upper limit on range for the system being tested of 40-m separation. This implies an attenuation length of approximately 5 m is reasonable for 385 nm light, although a bit higher than the expected value of 10 m to 20 m.

Daylight testing of the optical modem from the Atlantis CTD rosette has demonstrated the feasibility of blocking background light from solar irradiance, vehicle lighting, or other secondary emissions. Emitter wavelength selection of 385 nm and optical blocking filter UG1 provide a reasonable solution for optical link extent and background light extinction. Optical emitter and filter configurations can be refined to optimize system performance over various link separations and background light conditions.

After reviewing the present disclosure, those skilled in the art will know or be able to ascertain using no more than routine experimentation, many equivalents to the embodiments and practices described herein. For example, the illustrative embodiments discuss the use of UUVs, but other underwater vehicles such as remotely operated vehicles (ROVs) and autonomous underwater vehicles (AUVs), gliders, as well as submersibles carrying one or more humans, may be used with the systems and methods described herein. Accordingly, it will be understood that the systems and methods described are not to be limited to the embodiments disclosed herein, but is to be understood from the following claims, which are to be interpreted as broadly as allowed under the law.

Although specific features of the present invention are shown in some drawings and not in others, this is for convenience only, as each feature may be combined with any or all of the other features in accordance with the invention. While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature.

It is the intention, therefore, to be limited only as indicated by the scope of the claims appended hereto. Other embodiments will occur to those skilled in the art and are within the following claims.

What is claimed is:

1. A system for reducing fouling of a surface subjected to an aquatic environment, the system comprising:
a surface designed to be subjected to an aquatic environment;
a light source for emitting UV radiation toward the surface in a first pattern;
a pressure vessel;
an end cap adapted to provide at least one of a mechanical connection and an electrical connection to an object; and
control circuitry for driving the light source;
wherein the light source is disposed within the pressure vessel, and the pressure vessel is capable of passing the UV radiation emitted from the light source to the surface to reduce fouling.

2. The system of claim 1, wherein the emitted UV radiation is within the wavelength range of approximately 240 nm to 295 nm.

3. The system of claim 2, wherein the emitted UV radiation is within the wavelength range of approximately 250 nm to 260 nm.

4. The system of claim 1, wherein the control circuitry is adapted to maintain a constant duty cycle of the light source of at least about ten percent.

5. The system of claim 4 further comprising a microprocessor and a built-in duty cycle timer to minimize power consumption wherein the duty cycle timer consumes little to no power when no power is provided to the control circuitry.

6. The system of claim 1, wherein the pressure vessel is adapted to operate up to a depth of 500 m or more.

7. The system of claim 1, wherein the light source is selected from a fluorescent lamp and a LED.

8. The system of claim 1, wherein the pressure vessel retains a gas selected from atmospheric air, an inert gas, nitrogen gas, and a combination thereof.

9. The system of claim 1 further comprising a configurable optical reflector capable of tailoring the UV emission to at least one of a wider angle pattern and a narrower angle pattern relative to the first pattern.

10. The system of claim 1, wherein the object is selected from the group comprising a node, an observatory, a transducer, an optical modem, a vehicle, an AUV, an ROV, an UUV, a winch, a dock, and a profiler.

11. The system of claim 1, wherein the surface is selected from the group comprising a cable, a winch, a spool, a fin, a propeller, a light, a sensor, a transducer, an optically transparent surface, a window, a camera window, a lens, and a surface unsuitable for an antifouling coating.

12. The system of claim 1, wherein the system is capable of reducing fouling of the surface when disposed a distance from said surface of up to 30 cm or more.

13. A system for reducing the fouling of a UV-transmissive surface subjected to an aquatic environment, the system comprising:
a surface designed to be subjected to an aquatic environment;
a watertight housing;
a plurality of mounts disposed within the housing and proximate to the surface, each mount comprising an LED for emitting UV radiation; and
control circuitry for driving the LED and maintaining a duty cycle;
wherein the UV radiation is transmitted from within the housing to the surface to reduce the fouling of said surface, and the UV radiation is within the wavelength range of about 265 nm and about 295 nm, and the control circuitry maintains a duty cycle of at least 10%.

14. The system of claim 13, wherein the surface is selected from the group comprising an optically transparent surface, a UV transparent material, a window, a camera window, a lens, a light, a sensor, and a surface unsuitable for an antifouling coating.

15. The system of claim 13, wherein an attenuated dosage reaching the surface is at least about 0.5 kJ/m².

16. The system of claim 13, wherein a kill efficiency at the surface is at least about 95%.

17. A method for reducing fouling of a surface subjected to an aquatic environment, the method comprising the steps of:
selecting a surface designed to be subjected to an aquatic environment;
providing a system comprising a light source for emitting UV radiation disposed within a pressure vessel;
driving the light source to emit radiation;
directing emitted UV radiation toward the surface; and
maintaining a duty cycle of at least about 10%.

18. The method of claim 17, wherein the surface is selected from the group comprising a cable, a winch, a spool, a fin, a propeller, a light, a sensor, a transducer, an optically transparent surface, a window, a camera window, a lens, and a surface unsuitable for an antifouling coating.

19. The method of claim 17, wherein the emitted UV radiation is within the wavelength range of approximately 240 nm to 295 nm.

20. The method of claim 19, wherein the emitted UV radiation is within the wavelength range of approximately 250 nm to 260 nm.

21. The method of claim 17 further comprising the step of disposing the system a distance from the surface of up to 30 cm or more to reduce the fouling of said surface.

22. The method of claim 17, wherein the emitted UV radiation is directed toward a surface by an optical reflector adapted to provide uniform illumination over the surface.
